(19) United States
(12) Patent Application Publication
(10) Pub. No.: US 2020/0105375 A1
(43) Pub. Date: Apr. 2, 2020

(54) MODELS FOR TARGETED SEQUENCING OF RNA

(71) Applicant: Grail, Inc., Menlo Park, CA (US)

(72) Inventors: WENYING PAN, Menlo Park, CA (US); HYUNSUNG JOHN KIM, San Francisco, CA (US); MATTHEW H. LARSON, San Francisco, CA (US); ALEXANDER W. BLOCKER, Mountain View, CA (US); EARL HUBBELL, Palo Alto, CA (US); ARASH JAMSHIDI, Redwood City, CA (US)

(21) Appl. No.: 16/584,936

(22) Filed: Sep. 26, 2019

Related U.S. Application Data
(60) Provisional application No. 62/738,949, filed on Sep. 28, 2018.

(51) Int. Cl. G16B 30/10; G16B 20/20; G16B 30/20; G16B 40/00

(52) U.S. Cl. CPC G16B 30/10; G16B 40/00; G16B 30/20; G16B 20/20

(57) ABSTRACT

Systems and methods for processing sequencing data of ribonucleic acid (RNA) molecules from a test sample include obtaining a plurality of sequence reads each derived from a RNA molecule obtained from the test sample, filtering the plurality of sequence reads, identifying one or more candidate variants from the filtered plurality of sequence reads, determining a quality score for each of the identified one or more candidate variants, the quality score indicating a likelihood that the candidate variant is a false positive detection of a mutation in the RNA molecule, and outputting the one or more candidate variants having a quality score greater than a threshold quality score.

This application claims the benefit of priority to U.S. Provisional Patent Application No. 62/738,949, filed on Sep. 28, 2018, and entitled “Models for Targeted Sequencing of RNA,” the contents of which are herein incorporated by reference in their entirety for all purposes.

### BACKGROUND

### 1. Field of Art

This disclosure generally relates to targeted sequencing and more specifically to using cell free RNA in variant calling and quality control.

### 2. Introduction

Analysis of circulating cell free nucleotides, such as cell free ribonucleic acid (cfRNA) or cell free deoxyribonucleic acid (cfDNA), using next generation sequencing (NGS) is recognized as a valuable tool for detection and diagnosis of cancer or other diseases. Identifying rare variants indicative of cancer using NGS requires deep sequencing of nucleotide sequences from a biological sample such as a tissue biopsy or blood drawn from a subject. Detecting DNA or RNA that originated from tumor cells from a blood sample is difficult because circulating tumor DNA (ctDNA) or circulating tumor RNA (ctRNA) is typically present at low levels relative to other molecules in cfDNA or cfRNA extracted from the blood. The inability of existing methods to identify true positives (e.g., indicative of cancer in the subject) from signal noise diminishes the ability of known and future systems to distinguish true positives from false positives caused by noise sources, which can result in unreliable results for variant calling or other types of analyses. Moreover, errors introduced during sample preparation and sequencing can make accurate identification of rare variants difficult.

A number of different methods have been developed for detecting variants, such as single nucleotide variants (SNVs), in sequencing data. Most conventional methods have been developed for calling variants from DNA or RNA sequencing data obtained from a tissue sample. These methods may not be suitable for calling variants from deep sequencing data obtained from a cell free nucleotide sample.

For non-invasive diagnostic and monitoring of cancer, targeted sequencing data of cell free nucleotides serve as an important bio-source. However, detection of variants in deep sequencing data sets poses distinct challenges: the number of sequenced fragments tend to be several order of magnitude larger (e.g., sequencing depth can be 2,000× or more), debilitating most of the existing variant callers in compute-time and memory usage.

### SUMMARY

In various embodiments, a method for processing sequencing data of ribonucleic acid (RNA) molecules from a test sample comprises obtaining a plurality of sequence reads each derived from a RNA molecule obtained from the test sample. The method further comprises filtering the plurality of sequence reads. The method further comprises identifying one or more candidate variants from the filtered plurality of sequence reads. The method further comprises determining a quality score for each of the identified one or more candidate variants, where the quality score indicating a likelihood that the candidate variant is a false positive detection of a mutation in the RNA molecule. The method further comprises outputting the one or more candidate variants having a quality score greater than a threshold quality score.

In some embodiments, obtaining the plurality of sequence reads comprises obtaining the test sample from an individual, where the test sample comprising a plurality of RNA molecules; preparing a RNA sequencing library from the plurality of RNA molecules; and generating the plurality of sequence reads from the RNA sequencing library.

In some embodiments, the sequencing library is enriched for one or more targeted RNA molecules prior to obtaining the plurality of sequence reads.

In some embodiments, the plurality of sequence reads are obtained (e.g., from a binary sequence alignment map (BAM) format file) using next-generation sequencing of the RNA sequencing library.

In some embodiments, the plurality of RNA molecules are labeled. In some embodiments, the plurality of RNA molecules are RNA transcripts.

In some embodiments, filtering the plurality of sequence reads comprises filtering at least one sequence read of the plurality of sequence reads having a least a threshold number of continuous nucleotide base mutations. Filtering may also include filtering at least one sequence read of the plurality of sequence reads having at least a threshold depth. Filtering may also include filtering out a number of leading nucleotide bases of at least one sequence read of the plurality of sequence reads. In some embodiments, the threshold number of continuous nucleotide base mutations is at least three, the threshold depth is at least 50,000, and/or the number of leading nucleotide bases is six.

In some embodiments, filtering the plurality of sequence reads comprises filtering at least one sequence read of the plurality of sequence reads having at least a threshold depth. In some embodiments, the threshold depth is at least 10,000, at least 20,000, at least 30,000, at least 40,000, or at least 50,000.

In some embodiments, filtering the plurality of sequence reads comprises removing a number of leading nucleotide bases of at least one sequence read of the plurality of sequence reads. In some embodiments, the number is four, five, six, seven, or eight.

In some embodiments, the threshold quality score is determined by performing calibration using a plurality of calibration samples, where each calibration sample including one or more control RNA molecules and a plurality of RNA molecules from one or more individuals.

In some embodiments, performing the calibration using calibration samples comprises, for each of the plurality of calibration samples, determining a depth of the calibration sample; and determining a sensitivity of the calibration sample, the sensitivity indicating a likelihood of detecting false positive mutations in the calibration sample. In some embodiments, for each of the plurality of calibration samples, the method further comprises determining an alternate frequency of the calibration sample.

In some embodiments, the method further comprises determining a value of the threshold quality score and modifying the value of the threshold quality score according to the performed calibration.

In some embodiments, determining the quality score for a candidate variant comprises accessing a plurality of parameters including a dispersion parameter r and a mean rate parameter m specific to the candidate variant, the r and m having been derived using a model; inputting read information of the plurality of sequence reads into a function parameterized by the plurality of parameters; and determining the quality score for the candidate variant using an output of the function based on the input read information.

In some embodiments, the plurality of parameters represent mean and shape parameters of a gamma distribution. The function may be a negative binomial based on the plurality of sequence reads and the plurality of parameters.

In some embodiments, the plurality of parameters represent parameters of a distribution that encodes an uncertainty level of nucleotide mutations with respect to a given position of a sequence read.

In some embodiments, a gamma distribution is one component of a mixture of the distribution.

In some embodiments, the plurality of parameters are derived from a training sample of sequence reads from a plurality of healthy individuals. In some embodiments, the training sample excludes a subset of the sequence reads from the plurality of healthy individuals based on filtering criteria.

In some embodiments, the filtering criteria indicates to exclude sequence reads that have (i) a depth less than a threshold value or (ii) an allele frequency greater than a threshold frequency. In some embodiments, the filtering criteria varies based on positions of candidate variants in a genome.

In some embodiments, the plurality of parameters are derived using a Bayesian Hierarchical model. In some embodiments, the Bayesian Hierarchical model includes a multinomial distribution grouping positions of sequence reads into latent classes. In some embodiments, the Bayesian Hierarchical model includes fixed covariates unrelated to training samples from healthy individuals. In some embodiments, the covariates are based on a plurality of nucleotides adjacent to a given position of a sequence read. In some embodiments, the covariates are based on a level of uniqueness of a given sequence read relative to a target region of a genome.

In some embodiments, the Bayesian Hierarchical model is estimated using a Markov chain Monte Carlo method. In some embodiments, the Markov chain Monte Carlo method uses a Metropolis-Hastings algorithm. In some embodiments, the Markov chain Monte Carlo method uses a Gibbs sampling algorithm. In some embodiments, the Markov chain Monte Carlo method uses Hamiltonian mechanics.

In some embodiments, the sequence read information includes a depth d of the plurality of sequence reads, and the function is parameterized by m·d.

In some embodiments, the quality score is a Phred-scaled likelihood.

In some embodiments, the plurality of sequence reads are obtained from sequencing cell free RNA molecules obtained from a test sample. In some embodiments, the method further comprises collecting or having collected the test sample from a blood sample of the individual and enriching a set of target cell free RNA molecules from the test sample and sequencing the set of target cell free RNA molecules to generate the plurality of sequence reads.

In some embodiments, the plurality of sequence reads are obtained from sequencing RNA molecules from a sample of blood, whole blood, plasma, serum, urine, cerebrospinal fluid, fecal, saliva, tears, a tissue biopsy, pleural fluid, pericardial fluid, or peritoneal fluid of an individual.

In some embodiments, the plurality of sequence reads are obtained from sequencing RNA molecules from tumor cells obtained from a tumor biopsy.

In some embodiments, the plurality of sequence reads are obtained from sequencing a RNA molecules from an isolate of cells from blood, the isolate of cells including at least buffy coat white blood cells or CD4+ cells.

In some embodiments, the method further comprises determining that the candidate variant is a false positive mutation by comparing the quality score to a threshold quality score. In some embodiments, the candidate variant is a single nucleotide variant.

In some embodiments, the model encodes noise levels of nucleotide mutations for one base of A, U, C, and G to each of the other three bases. In some embodiments, the candidate variant is an insertion or deletion of at least one nucleotide.

In some embodiments, the model includes a distribution of lengths of insertions or deletions.

In some embodiments, the model separates an inference for determining a likelihood of an alternate allele from an inference for determining a length of the alternate allele using the distribution of lengths.

In some embodiments, the distribution of lengths comprises a multinomial with Dirichlet prior. In an embodiment, the Dirichlet prior on the multinomial distribution of lengths is determined by covariates of anchor positions of a genome.

In some embodiments, the model includes a distribution ω determined based on covariates. In some embodiments, the model includes a distribution φ determined based on covariates and anchor positions of a genome.

In some embodiments, the model includes a multinomial distribution grouping lengths of insertions or deletions at anchor positions of sequence reads into latent classes. In an embodiment, an expected mean total count of insertions or deletions at a given anchor position is modeled by a distribution based on covariates and anchor positions of a genome.

In various embodiments, a method for processing ribonucleic acid (RNA) samples comprises obtaining a calibration sample including one or more control RNA molecules and a plurality of RNA molecules of an individual. The method further comprises determining sensitivity of detecting candidate variants in the calibration sample. The method further comprises generating a mapping of depth of the calibration sample to the sensitivity. The method further comprises determining candidate variants of one or more RNA molecules using the mapping.

In various embodiments, a method for processing ribonucleic acid (RNA) molecule comprises obtaining a plurality of calibration samples including one or more control RNA molecules and a RNA molecule from an individual, the plurality of calibration samples having different depths. The method further comprises generating mappings of the plurality of calibration samples, the mappings each indicating a likelihood of detecting false positive mutations in the RNA molecule and associated with the depth of a corresponding calibration sample. The method further comprises determining a depth of the RNA molecule. The method further comprises determining candidate variants of the RNA molecule using the mappings and the depth.

In some embodiments, a system comprises a computer processor and a memory, the memory storing instructions that when executed by the computer processor cause the processor to carry out the steps of any of the preceding paragraphs. In some embodiments, a non-transitory computer-readable storage medium stores instructions that when executed by a processor cause the processor to carry out the steps of any of the preceding paragraphs.

### BRIEF DESCRIPTION OF DRAWINGS

FIG. 1A is flowchart of a method for preparing a nucleic acid sample for sequencing according to one embodiment.

FIG. 1B is a graphical representation of a process for enriching targeted nucleic acid molecules according to one embodiment.

FIG. 2 is block diagram of a processing system for processing sequence reads according to one embodiment.

FIG. 3A is flowchart of a method for determining variants of sequence reads according to one embodiment.

FIG. 3B is flowchart of a method for determining variants of sequence reads of RNA according to one embodiment.

FIG. 3C is flowchart of a method for generating RNA calibration data according to one embodiment.

FIG. 3D is flowchart of a method for determining candidate variants based on sensitivity and depth of RNA sequence reads according to one embodiment.

FIG. 4 is a diagram of an application of a Bayesian hierarchical model according to one embodiment.

FIG. 5A shows dependencies between parameters and sub-models of a Bayesian hierarchical model for determining true single nucleotide variants according to one embodiment.

FIG. 5B shows dependencies between parameters and sub-models of a Bayesian hierarchical model for determining true insertions or deletions according to one embodiment.

FIGS. 6A and 6B illustrate diagrams associated with a Bayesian hierarchical model according to one embodiment.

FIG. 7A is a diagram of determining parameters by fitting a Bayesian hierarchical model according to one embodiment.

FIG. 7B is a diagram of using parameters from a Bayesian hierarchical model to determine a likelihood of a false positive according to one embodiment.

FIG. 8 is flowchart of a method for training a Bayesian hierarchical model according to one embodiment.

FIG. 9 is flowchart of a method for scoring candidate variants of a given nucleotide mutation according to one embodiment.

FIGS. 10A and 10B are diagrams comparing the number of cfDNA and cfRNA and the alternate frequency (AF) of cfDNA and cfRNA variants detected without using a noise model.

FIG. 11 is a diagram showing the number of variants detected at various threshold quality scores according to one embodiment.

FIG. 12 is a diagram of observed and theoretical quality scores according to one embodiment.

FIG. 13 is a diagram showing the distribution of quality scores for calibration samples of RNA according to one embodiment.

FIG. 14 is a diagram showing depth distributions observed for control RNA molecules according to one embodiment.

FIGS. 15A and 15B are diagrams of sensitivity based on depth and alternate frequency of calibration samples of RNA according to various embodiments.

FIGS. 16A and 16B are diagrams showing the number of variants detected in cfRNA samples based on type and stage of cancer according to various embodiments.

FIG. 17 is a diagram showing different types of variants detected in cfRNA samples according to one embodiment.

FIGS. 18A and 18B are diagrams showing comparisons of variant detection rates in cfDNA and cfRNA samples according to various embodiments.

FIG. 19 shows a schematic of an example computer system for implementing various methods of the present invention.

The figures depict embodiments of the present invention for purposes of illustration only. One skilled in the art will readily recognize from the following discussion that alternative embodiments of the structures and methods illustrated herein may be employed without departing from the principles of the invention described herein.

### DETAILED DESCRIPTION

Reference will now be made in detail to several embodiments, examples of which are illustrated in the accompanying figures. It is noted that wherever practicable similar or like reference numbers may be used in the figures and may indicate similar or like functionality. For example, a letter after a reference numeral, such as “sequence reads 180A,” indicates that the text refers specifically to the element having that particular reference numeral. A reference numeral in the text without a following letter, such as “sequence reads 180,” refers to any or all of the elements in the figures bearing that reference numeral (e.g. “sequence reads 180” in the text refers to reference numerals “sequence reads 180A” and/or “sequence reads 180B” in the figures).

### I. Definitions

The term “individual” refers to a human individual. The term “healthy individual” refers to an individual presumed to not have a cancer or disease. The term “subject” refers to an individual who is known to have, or potentially has, a cancer or disease.

The term “sequence reads” refers to nucleotide sequences read from a sample obtained from an individual. Sequence reads can be obtained through various methods known in the art.

The term “read segment” or “read” refers to any nucleotide sequences including sequence reads obtained from an individual and/or nucleotide sequences derived from the initial sequence read from a sample obtained from an individual. For example, a read segment can refer to an aligned sequence read, a collapsed sequence read, or a stitched read. Furthermore, a read segment can refer to an individual nucleotide base, such as a single nucleotide variant.

The term “single nucleotide variant” or “SNV” refers to a substitution of one nucleotide to a different nucleotide at a position (e.g., site) of the nucleotide in a sequence, e.g., a sequence read from an individual. A substitution from a first nucleobase “X” to a second nucleobase “Y” may be denoted as “X>Y.” For example, a cytosine to thymine SNV may be denoted as “C>T.”

The term “indel” refers to any insertion or deletion of one or more bases having a length and a position (which may also be referred to as an anchor position) in a sequence read. An insertion corresponds to a positive length, while a deletion corresponds to a negative length.

The term “mutation” refers to one or more SNVs or indels.

The term “true positive” refers to a SNV or mutation that indicates real biology, for example, presence of a potential cancer, disease, or germline mutation in an individual. True positives are not caused by mutations naturally occurring in healthy individuals (e.g., recurrent mutations) or other sources of artifacts such as process errors during assay preparation of nucleic acid samples.

The term “false positive” refers to a mutation incorrectly determined to be a true positive. Generally, false positives may be more likely to occur when processing sequence reads associated with greater mean noise rates or greater uncertainty in noise rates.

The term “cell free nucleic acid,” “cell free DNA,” “cfDNA,” “cell free RNA,” or “cfRNA” refers to nucleic acid fragments that circulate in an individual's body (e.g., bloodstream) and originate from one or more healthy cells and/or from one or more cancer cells.

The term “circulating tumor DNA” or “ctDNA” refers to deoxyribonucleic acid fragments that originate from tumor cells or other types of cancer cells, which may be released into an individual's bloodstream, for example, as result of biological processes such as apoptosis or necrosis of dying cells or actively released by viable tumor cells. The term “circulating tumor RNA” or “ctRNA” refers to ribonucleic acid fragments that originate from tumor cells or other types of cancer cells, which may be released into an individual's bloodstream, for example, as result of biological processes such as apoptosis or necrosis of dying cells or actively released by viable tumor cells.

The term “genomic nucleic acid,” “genomic DNA,” or “gDNA” refers to nucleic acid including chromosomal DNA that originate from one or more healthy cells.

The term “alternative allele” or “ALT” refers to an allele having one or more mutations relative to a reference allele, e.g., corresponding to a known gene.

The term “sequencing depth” or “depth” refers to a total number of sequence reads or read segments derived from the same position or location of the genome from a sample.

The term “alternate depth” or “AD” refers to a number of sequence reads or read segments in a sample that support an ALT, e.g., include mutations of the ALT.

The term “reference depth” refers to a number of sequence reads or read segments in a sample that include a reference allele at a candidate variant location.

The term “alternate frequency” or “AF” refers to the frequency of a given ALT. The AF may be determined by dividing the corresponding AD of a sample by the depth of the sample for the given ALT.

The term “variant” or “true variant” refers to a SNV or mutated nucleotide base at a position in the genome. Such a variant can be indicative or, or may lead to, the development and/or progression of cancer in an individual.

The term “edge variant” refers to a mutation located near an edge of a sequence read, for example, within a threshold distance of nucleotide bases from the edge of the sequence read.

The term “candidate variant,” “called variant,” “putative variant,” or refers to one or more detected nucleotide variants of a nucleotide sequence, for example, at a position in the genome that is determined to be mutated. Generally, a nucleotide base is deemed a called variant based on the presence of an alternative allele on sequence reads obtained from a sample, where the sequence reads each cross over the position in the genome. The source of a candidate variant may initially be unknown or uncertain. During processing, candidate variants may be associated with an expected source such as gDNA (e.g., blood-derived) or cells impacted by cancer (e.g., tumor-derived). Additionally, candidate variants may be called as true positives.

The term “non-edge variant” refers to a candidate variant that is not determined to be resulting from an artifact process, e.g., using an edge variant filtering method described herein. In some scenarios, a non-edge variant may not be a true variant (e.g., mutation in the genome) as the non-edge variant could arise due to a different reason as opposed to one or more artifact processes.

### II. Example Assay Protocol

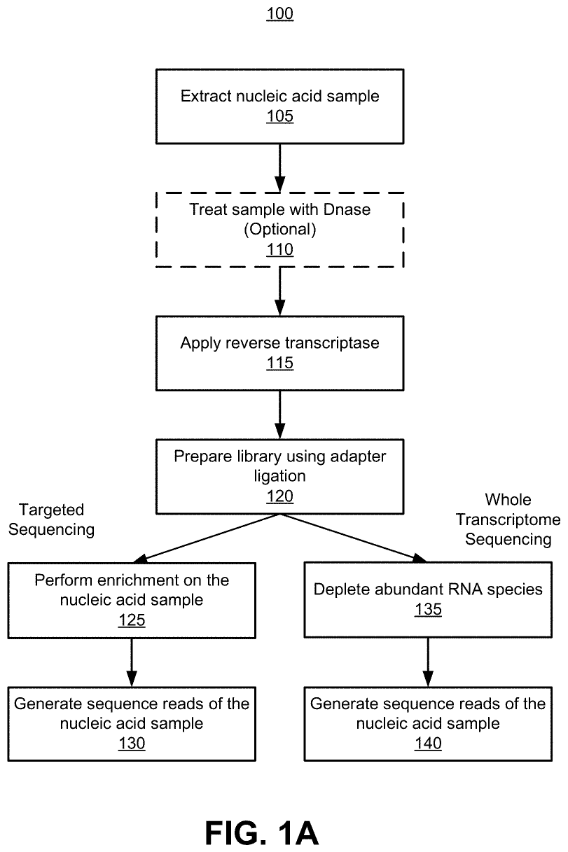

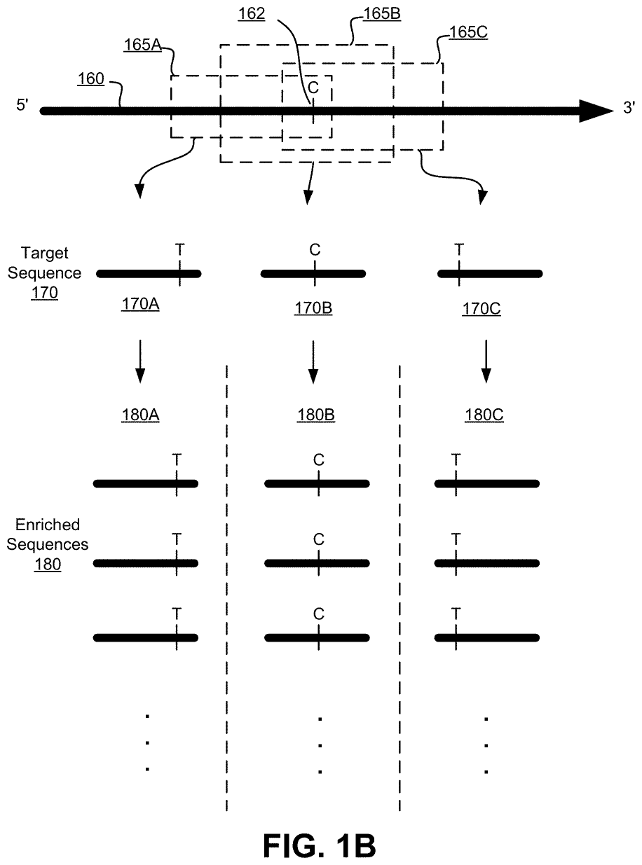

FIG. 1A is flowchart of a method 100 for preparing a nucleic acid sample for sequencing according to one embodiment. The method 100 includes, but is not limited to, the following steps. For example, any step of the method 100 may comprise a quantitation sub-step for quality control or other laboratory assay procedures known to one skilled in the art.

In step 105, a ribonucleic nucleic acid (RNA) or deoxyribonucleic nucleic acid (DNA) sample is extracted from a test sample from a subject. The following embodiments for using error source information in variant calling and quality control may be applicable to one or both of DNA and RNA types of nucleic acid sequences. However, the examples described herein may focus on samples including RNA molecules for purposes of clarity and explanation. The sample may be any subset of the human genome, including the whole genome. Alternatively, the sample may be any subset of the human transcriptome, including the whole transcriptome. The sample may be extracted from a subject known to have or suspected of having cancer, or from a healthy subject. The sample may include blood, plasma, serum, urine, fecal, saliva, other types of bodily fluids, or any combination thereof. In some embodiments, methods for drawing a blood sample (e.g., syringe or finger prick) may be less invasive than procedures for obtaining a tissue biopsy, which may require surgery. The extracted sample may comprise cfRNA or cfDNA.

Steps 110-115 may be performed for preparation of a sample targeting RNA molecules. In other embodiments, steps 110-115 may not be performed for preparation of a different sample targeting DNA molecules. In step 110, the nucleic acid sample including RNA molecules is optionally treated with a DNase enzyme. The DNase may remove DNA molecules from the nucleic acid sample to reduce DNA contamination of the RNA molecules. After RNA molecules are converted into DNA, it may be difficult to distinguish the RNA-converted DNA and genomic DNA originally found in the nucleic acid sample. Applying the DNase allows for targeted amplification of molecules originating from cfRNA. The DNase process may include steps for adding a DNase buffer, mixing the sample applied with DNase using a centrifuge, and incubation. In some embodiments, step 110 includes one or more processes based on the DNase treatment protocol described in the Qiagen QIAamp Circulating Nucleic Acid Handbook.

In step 115, a reverse transcriptase enzyme is applied to convert the RNA molecules in the nucleic acid sample into complementary DNA (cDNA). The reverse transcriptase process may include a first-strand synthesis step (conversion of RNA to cDNA via reverse transcription) and second-strand synthesis steps (conversion of cDNA to double strand DNA (dsDNA) using a polymerase). During first-strand synthesis, a primer anneals to the 3′ end of a RNA molecule. During second-strand synthesis, a different primer anneals to the 3′ end of the cDNA molecule.

In step 120, a sequencing library is prepared. During library preparation, unique molecular identifiers (UMI) are added to the nucleic acid molecules in the sample through adapter ligation. The UMIs are short nucleic acid sequences (e.g., 4-10 base pairs) that are added to one or both ends of nucleic acid fragments during adapter ligation. In some embodiments, UMIs are a short string of degenerate or semi-degenerate base pairs that serve as a unique tag that can be used to identify sequence reads originating from a specific nucleic acid molecule or fragment deriving from the sample. During PCR amplification following adapter ligation, the UMIs are replicated along with the attached nucleic acid molecule or fragment, which provides a way to identify amplicons, or subsequent sequence reads, that came from the same original fragment in downstream analysis.

For embodiments including targeted sequencing of RNA or DNA, in step 125, targeted nucleic acid sequences are enriched from the library. Targeted nucleic acids can be enriched using any known means in the art. For example, in one embodiment, during enrichment, hybridization probes (also referred to herein as “probes”) are used to target, and pull down, nucleic acid fragments informative for the presence or absence of cancer (or disease), cancer status, or a cancer classification (e.g., cancer type or tissue of origin). For a given workflow, the hybridization probes may be designed to anneal (or hybridize) to a target (complementary) strand of RNA or DNA. The target strand may be the “positive” strand (e.g., the strand transcribed into mRNA, and subsequently translated into a protein) or the complementary “negative” strand. The probes may range in length from 10 s, 100 s, or 1000 s of base pairs. In one embodiment, the probes are designed based on a gene panel to analyze particular mutations or target regions of the genome (e.g., of the human or another organism) that are suspected to correspond to certain cancers or other types of diseases. Moreover, in some embodiments the probes may be tiled to cover overlapping portions of a target region.

Additionally, for targeted sequencing, in step 130, sequence reads are generated from the enriched nucleic acid sample. Sequencing data may be acquired from the enriched RNA or DNA sequences by known means in the art. For example, the method 100 may include next generation sequencing (NGS) techniques including synthesis technology (Illumina), pyrosequencing (454 Life Sciences), ion semiconductor technology (Ion Torrent sequencing), single-molecule real-time sequencing (Pacific Biosciences), sequencing by ligation (SOLiD sequencing), nanopore sequencing (Oxford Nanopore Technologies), or paired-end sequencing. In some embodiments, massively parallel sequencing is performed using sequencing-by-synthesis with reversible dye terminators. Generation of sequence reads is further described below with reference to FIG. 1B.

In other embodiments, for example, in a whole transcriptome sequencing approach (e.g., instead of targeted sequencing), in step 135, abundant RNA species are depleted from the nucleic acid sample. For example, in some embodiments ribosomal RNA (rRNA) and/or transfer RNA (tRNA) species can be depleted. Available commercial kits, such as RiboMinus™ (ThermoFisher Scientific) or AnyDeplete (NuGen), can be used for depletion of abundant RNA species. In an embodiment, after depletion of abundant RNA species, sequence reads are generated in step 140.

FIG. 1B is a graphical representation of a process for enriching targeted nucleic acid molecules according to one embodiment. FIG. 1B depicts one example of a nucleic acid segment 160 (i.e., a target segment or region) from the genome of a subject. The illustrated example depicts three regions 165A, 165B, and 165C of the nucleic acid segment 160 that can be targeted by different hybridization probes. More specifically, hybridization probes complementary to each of the three regions 165A, 165B, and 165C can be used to target, and thus, enrich for nucleic acids corresponding to each of the three regions. As shown, each of the three regions 165A, 165B, and 165C overlaps a base position 162 on the nucleic acid segment 160. The cytosine nucleotide base 162 is located near a first edge of region 165A, at the center of region 165B, and near a second edge of region 165C.

In some embodiments, one or more (or all) of the probes are designed based on a gene panel to analyze particular mutations or target regions of the genome (e.g., of the human or another organism) that are suspected to correspond to certain cancers or other types of diseases. For example, as shown in FIG. 1B, the targeted base position cytosine (“C”), could be a position where an SNV may occur that is known to be associated with cancer. By using a targeted gene panel rather than sequencing all expressed genes of a genome, also known as “whole exome sequencing” or “whole transcriptome sequencing,” the method 100 may be used to increase sequencing depth of the target regions, where depth refers to the count of the number of times a given target sequence within the sample has been sequenced. Increasing sequencing depth reduces required input amounts of the nucleic acid sample and can lower sequencing costs.

Hybridization of nucleic acids corresponding to, or derived from, the targeted nucleic acid segment 160 using one or more probes results in an enrichment of one or more target sequence 170 derived from the nucleic acid segment 160 (i.e., the target segment or region). As shown in FIG. 1B, the enriched target sequence 170 is the nucleotide base sequence derived from the region 165 that is targeted by a hybridization probe. As shown, enriched target sequence 170A corresponds to region 165A targeted by a first hybridization probe, enriched target sequence 170B corresponds to region 165B targeted by a second hybridization probe, and enriched target sequence 170C corresponds to region 165C targeted by a third hybridization probe. The hybridization probes, each enriched target sequence 170A, 170B, and 170C includes a nucleotide base that corresponds to the cytosine nucleotide base 162 being targeted for analysis on the target segment 160.

In the example of FIG. 1B, the enriched target sequence 170A and enriched target sequence 170C each have a nucleotide base (shown as thymine “T”) that is located near the edge of the enriched target sequences 170A and 170C. Thus, the C>T SNV for enriched target sequences 170A and 170C may be considered an edge variant because the mutation is located at an edge of target sequences 170A and 170C. Here, the thymine nucleotide base (e.g., as opposed to a cytosine base) may be a result of a random cytosine deamination process (which tends to occur more frequently near the edges of nucleic acid fragments), and results in the cytosine base to be subsequently recognized as a thymine nucleotide base during the sequencing process. Because random cytosine deamination tends to be more prevalent near the edge of an enriched target nucleic acid, the detected edge variants (in this case thymine) may be false positives. In contrast, enriched target sequence 170B has a cytosine base that is located at the center of the enriched target sequence 170B. Here, because the cytosine base is located closer to the center of the targeted sequence, it is likely a correct call at that position (i.e., the base located at that position in the nucleic acid molecule being tested) because the position is less susceptible to random cytosine deamination. If the correct call matches the expected base in the reference (e.g., as shown at 160), then no SNV is detected. However, if the called base at that position does not match the reference, the called base is likely an SNV.

After enrichment, the enriched nucleic acid fragments may be amplified using PCR. For example, the enriched target sequences 170 can be amplified to obtain a plurality of amplicons 180 that can be subsequently sequenced. In some embodiments, each of the amplicons 180 is replicated from enriched target sequence 170. Amplicons 180A and 180C that are amplified from enriched target sequences 170A and 170C, respectively, also include the thymine nucleotide base located near the edge of each sequence read 180A or 180C. As used hereafter, the mutated nucleotide base (e.g., thymine nucleotide base) in the enriched sequence 180 that is mutated in relation to the reference allele (e.g., cytosine nucleotide base 162) is considered as the alternative allele. Additionally, each amplicon sequence 180B amplified from enriched target sequence 170B includes the cytosine nucleotide base located near or at the center of each amplicon sequence 180B.

In some embodiments, the sequence reads may be aligned to a reference genome using known methods in the art to determine alignment position information. The alignment position information may indicate a beginning position and an end position of a region in the reference genome that corresponds to a beginning nucleotide base and end nucleotide base of a given sequence read. Alignment position information may also include sequence read length, which can be determined from the beginning position and end position. A region in the reference genome may be associated with a gene or a segment of a gene.

In various embodiments, a sequence read is comprised of a read pair denoted as R₁ and R₂. For example, the first read R₁ may be sequenced from a first end of a nucleic acid fragment whereas the second read R₂ may be sequenced from the second end of the nucleic acid fragment. Therefore, nucleotide base pairs of the first read R₁ and second read R₂ may be aligned consistently (e.g., in opposite orientations) with nucleotide bases of the reference genome. Alignment position information derived from the read pair R₁ and R₂ may include a beginning position in the reference genome that corresponds to an end of a first read (e.g., R₁) and an end position in the reference genome that corresponds to an end of a second read (e.g., R₂). In other words, the beginning position and end position in the reference genome represent the likely location within the reference genome to which the nucleic acid fragment corresponds. An output file having SAM (sequence alignment map) format or BAM (binary) format may be generated and output for further analysis such as variant calling.

### III. Example Processing System

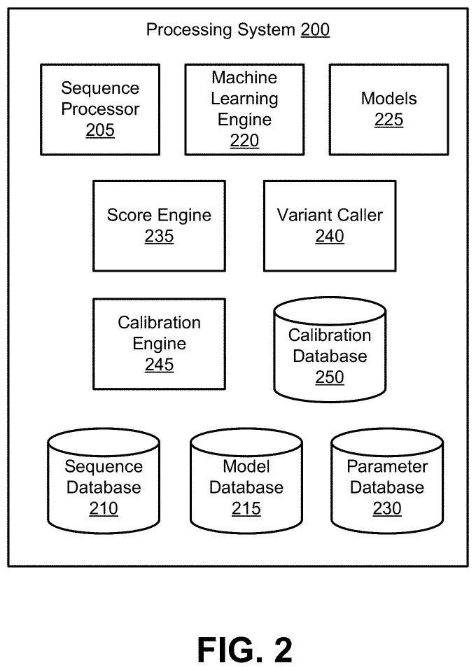

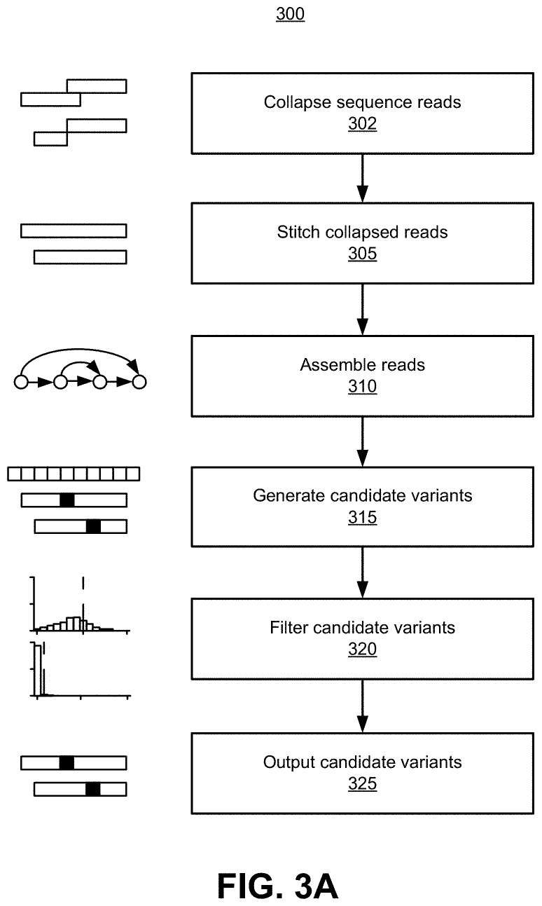

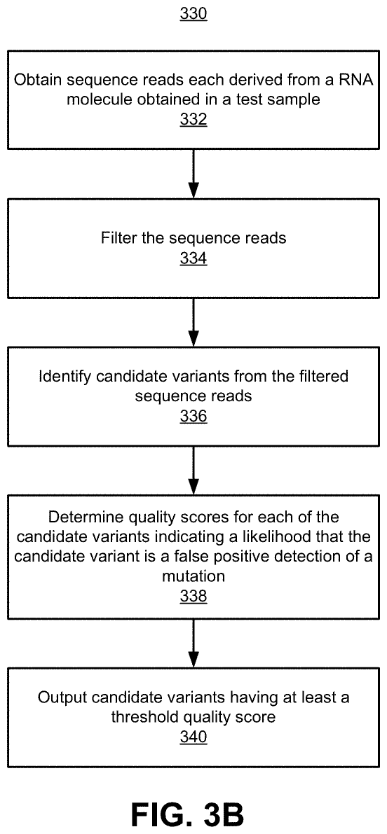

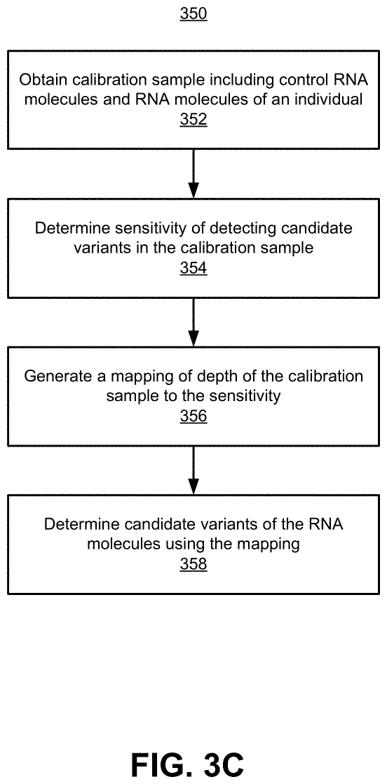

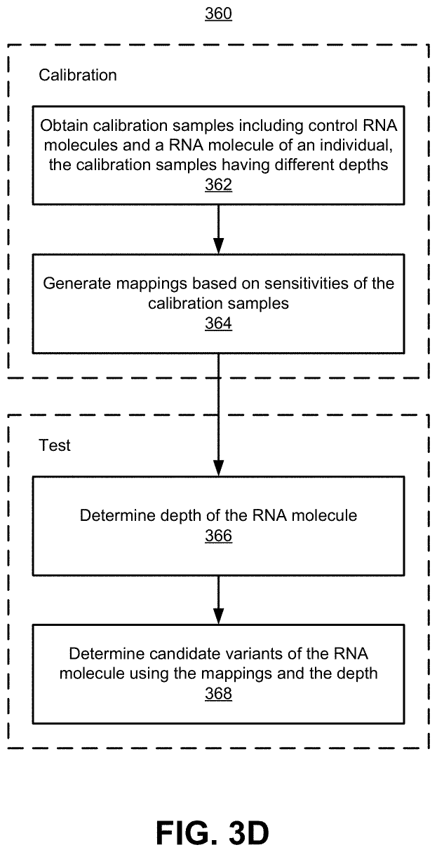

FIG. 2 is block diagram of a processing system 200 for processing sequence reads according to one embodiment. The processing system 200 includes a sequence processor 205, sequence database 210, model database 215, machine learning engine 220, models 225 (for example, including one or more Bayesian hierarchical models), parameter database 230, score engine 235, variant caller 240, calibration engine 245, and calibration database 250. FIG. 3A is flowchart of a method 300 for determining variants of sequence reads according to one embodiment. In some embodiments, the processing system 200 performs the method 300 to perform variant calling (e.g., for SNVs and/or indels) based on input sequencing data. Further, the processing system 200 may obtain the input sequencing data from an output file associated with nucleic acid sample prepared using the method 100 described above. The method 300 includes, but is not limited to, the following steps, which are described with respect to the components of the processing system 200. In other embodiments, one or more steps of the method 300 may be optional or replaced by a step of a different process for generating variant calls, e.g., using Variant Call Format (VCF), such as HaplotypeCaller, VarScan, Strelka, or SomaticSniper.

At step 302, the sequence processor 205 collapses aligned sequence reads of the input sequencing data. In one embodiment, collapsing sequence reads includes using UMIs, and optionally alignment position information from sequencing data of an output file (e.g., from the method 100 shown in FIG. 1) to collapse multiple sequence reads into a consensus sequence for determining the most likely sequence of a nucleic acid fragment or a portion thereof. The unique sequence tag can be from about 4 to 20 nucleic acids in length. Since the UMIs are replicated with the ligated nucleic acid fragments through enrichment and PCR, the sequence processor 205 may determine that certain sequence reads originated from the same molecule in a nucleic acid sample. In some embodiments, sequence reads that have the same or similar alignment position information (e.g., beginning and end positions within a threshold offset) and include a common UMI are collapsed, and the sequence processor 205 generates a collapsed read (also referred to herein as a consensus read) to represent the nucleic acid fragment. The sequence processor 205 designates a consensus read as “duplex” if the corresponding pair of collapsed reads have a common UMI, which indicates that both positive and negative strands of the originating nucleic acid molecule is captured; otherwise, the collapsed read is designated “non-duplex.” In some embodiments, the sequence processor 205 may perform other types of error correction on sequence reads as an alternate to, or in addition to, collapsing sequence reads. In embodiments for processing of samples including RNA molecules, collapsing of duplex reads is not required, e.g., because the RNA molecules are single stranded unlike double-stranded DNA molecules.

At step 305, the sequence processor 205 stitches the reads based on the corresponding alignment position information. In some embodiments, the sequence processor 205 compares alignment position information between a first read and a second read to determine whether nucleotide base pairs of the first and second reads overlap in a template, e.g., a reference genome or exon. In one use case, responsive to determining that an overlap (e.g., of a given number of nucleotide bases) between the first and second reads is greater than a threshold length (e.g., threshold number of nucleotide bases), the sequence processor 205 designates the first and second reads as “stitched”; otherwise, the reads are designated “unstitched.” In some embodiments, a first and second read are stitched if the overlap is greater than the threshold length and if the overlap is not a sliding overlap. For example, a sliding overlap may include a homopolymer run (e.g., a single repeating nucleotide base), a dinucleotide run (e.g., two-nucleotide repeating base sequence), or a trinucleotide run (e.g., three-nucleotide repeating base sequence), where the homopolymer run, dinucleotide run, or trinucleotide run has at least a threshold length of base pairs.

At step 310, the sequence processor 205 assembles reads into paths. In some embodiments, the sequence processor 205 performs assembly using the SAMtools software tool to generate a pileup format of the reads. In some embodiments, the sequence processor 205 assembles reads to generate a directed graph, for example, a de Bruijn graph, for a target region (e.g., a gene). Unidirectional edges of the directed graph represent sequences of k nucleotide bases (also referred to herein as “k-mers”) in the target region, and the edges are connected by vertices (or nodes). The sequence processor 205 aligns reads to a directed graph such that any of the reads may be represented in order by a subset of the edges and corresponding vertices. In one embodiment, the sequence processor 205 assembles reads from a DNA molecule sample based on a reference of a full gene or transcript. In other embodiments, the sequence processor 205 assembles reads from a RNA molecule sample based on a reference of an exon.

In some embodiments, the sequence processor 205 determines sets of parameters describing directed graphs and processes directed graphs. Additionally, the set of parameters may include a count of successfully aligned k-mers from reads to a k-mer represented by a node or edge in the directed graph. The sequence processor 205 stores, e.g., in the sequence database 210, directed graphs and corresponding sets of parameters, which may be retrieved to update graphs or generate new graphs. For instance, the sequence processor 205 may generate a compressed version of a directed graph (e.g., or modify an existing graph) based on the set of parameters. In one use case, in order to filter out data of a directed graph having lower levels of importance, the sequence processor 205 removes (e.g., “trims” or “prunes”) nodes or edges having a count less than a threshold value, and maintains nodes or edges having counts greater than or equal to the threshold value.

At step 315, the variant caller 240 generates candidate variants from the paths assembled by the sequence processor 205. In one embodiment, the variant caller 240 generates the candidate variants by comparing a directed graph (which may have been compressed by pruning edges or nodes in step 310) to a reference sequence of a target region of a genome. The variant caller 240 may align edges of the directed graph to the reference sequence, and records the genomic positions of mismatched edges and mismatched nucleotide bases adjacent to the edges as the locations of candidate variants. In some embodiments, the genomic positions of mismatched edges and mismatched nucleotide bases to the left and right of edges are recorded as the locations of called variants. Additionally, the variant caller 240 may generate candidate variants based on the sequencing depth of a target region. In particular, the variant caller 240 may be more confident in identifying variants in target regions that have greater sequencing depth, for example, because a greater number of sequence reads help to resolve (e.g., using redundancies) mismatches or other base pair variations between sequences.

In one embodiment, the variant caller 240 generate candidate variants using a model 225 to determine expected noise rates for sequence reads from a subject. The model 225 may be a Bayesian hierarchical model, though in some embodiments, the processing system 200 uses one or more different types of models. Moreover, a Bayesian hierarchical model may be one of many possible model architectures that may be used to generate candidate variants and which are related to each other in that they all model position-specific noise information in order to improve the sensitivity/specificity of variant calling. More specifically, the machine learning engine 220 trains the model 225 using samples from healthy individuals to model the expected noise rates per position of sequence reads.

Further, multiple different models 225 may be stored in the model database 215 or retrieved for application post-training. For example, a first model is trained to model SNV noise rates and a second model is trained to model indel noise rates. Additionally, one model may be trained to process RNA molecules and a different model may be trained to process DNA molecules. Further, the score engine 235 may use parameters of the model 225 to determine a likelihood of one or more true positives in a sequence read. The score engine 235 may determine a quality score (e.g., on a logarithmic scale) based on the likelihood. For example, the quality score is a Phred quality score Qual=−10·log₁₀P, where P is the likelihood of an incorrect candidate variant call (e.g., a false positive). Other models 225 may use output of one or more Bayesian hierarchical models to determine expected noise of nucleotide mutations in sequence reads of different samples.

At step 320, the processing system 200 filters the candidate variants using one or more types of models 225 or filters. For example, the score engine 235 scores the candidate variants using a noise model, edge variant prediction model, or corresponding likelihoods of true positives or quality scores. In addition, the processing system 200 may filter edge variants and/or non-synonymous mutations. In an embodiment, the processing system 200 filters out candidate variants that have less than a threshold quality score, e.g., determined by the score engine 235 or a model 225.

At step 325, the processing system 200 outputs the filtered candidate variants. In some embodiments, the processing system 200 outputs some or all of the determined candidate variants along with corresponding one or more quality scores from the filtering steps. Downstream systems, e.g., external to the processing system 200 or other components of the processing system 200, may use the candidate variants and quality scores for various applications including, but not limited to, predicting presence of cancer, disease, or germline mutations.

FIG. 3B is flowchart of a method 330 for determining variants of sequence reads of RNA according to one embodiment. The method 330 may be performed as an alternative to the method 300 shown in FIG. 3A. The method 300 may be used to process samples for sequence reads of DNA or RNA, while the method 330 may include certain steps for processing samples for sequence reads of RNA in particular. In step 332, sequence reads of a test sample are obtained, for instance, using one or more steps of the method 100 previously described with reference to FIG. 1. The sequence reads may each be derived from a RNA molecule obtained from the test sample and may be RNA transcripts, such as messenger RNA (mRNA), transfer RNA (tRNA) or ribosomal RNA (rRNA). In an embodiment, the RNA transcripts are mRNA molecules. The test sample may be obtained from an individual, for instance, by drawing a sample of blood or another type of bodily fluid of the individual. A sequencing library may be prepared from the RNA molecules, and the sequencing library may be enriched for targeted RNA molecules prior to generating the sequence reads. Alternatively, the blood sample (or extracted nucleic acid sample therefrom) can be enriched for targeted RNA molecules prior to preparation of the sequencing library. In some embodiments, the processing system 200 aligns the sequence reads to a DNA reference genome (e.g., the human genome assembly GRCh37 (hg19) from Genome Reference Consortium). In other embodiments, the processing system 200 aligns the sequence reads to a transcriptome reference based on a set of RNA molecules in one or more cells. The transcriptome may be obtained from the GENCODE dataset release 19 of the Genome Reference Consortium Human Build 37 (GRCh37). Harrow J, et al. (2012) GENCODE: The reference human genome annotation for The ENCODE Project. The sequence reads may be simultaneously aligned to the transcriptome and genome.

In some embodiments, the sequence reads are obtained from a binary sequence alignment map (BAM) format file or variant call format (VCF) file generated using next-generation sequencing of a sequencing library prepared from a sample comprising a plurality of RNA molecules or a plurality of RNA transcripts (e.g., mRNA). A BAM file is indexed at the level sequence reads, and the VCF file, indexed by variants, can be generated using the corresponding BAM file. The VCF file may include variant data aggregated across multiple sequence reads, which may provide for more efficient lookup of certain variant, in comparison to searching an index of sequence reads of a BAM file.

In step 334, the processing system 200 filters the sequence reads according to one or more criteria. As an example criteria, the processing system 200 filters sequence reads that have at least a threshold number (e.g., 3, 10, 100, 500, or more) of (e.g., identical) sequential nucleotide base mutations, which may be artifacts introduced during library preparation during generation of the sequence reads. The processing system 200 may filter sequence reads having at least a threshold depth (e.g., at least 10,000, at least 20,000, at least 30,000, at least 40,000, or at least 50,000 nucleotide bases or more). Sequences reads having at least the threshold depth may represent, for example, abundant transcripts that are ubiquitously expressed and/or did not properly collapse, and thus are removed from further processing. In an embodiment, the processing system 200 trims sequence reads by removing a predetermined number (e.g., 4, 5, 6, 7, or 8) of leading nucleotide bases from one or more of the sequence reads. The trimming may remove errors caused by random hexamer mismatches. The filtering steps may be effective in reducing noise of the sequence reads because RNA coverage has more variance than that of DNA.

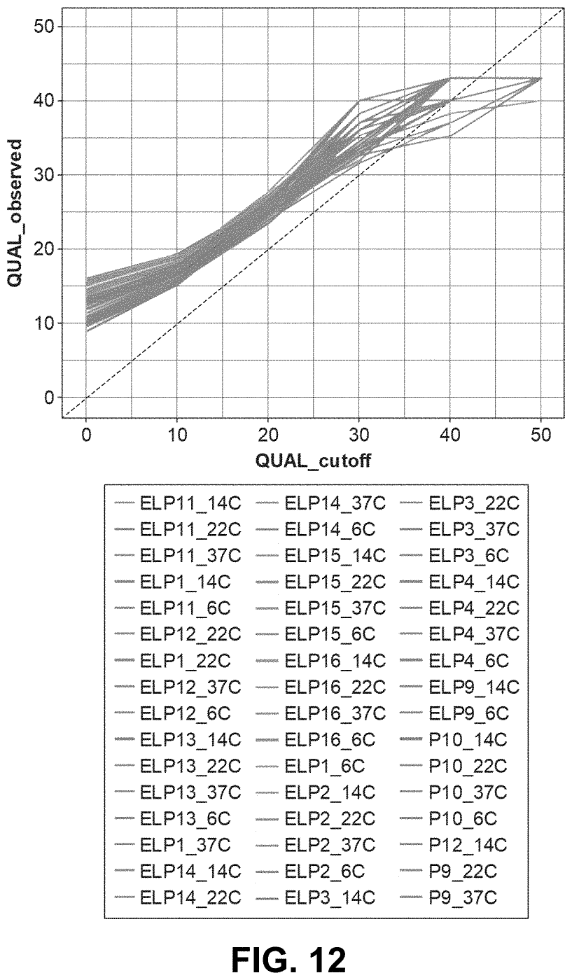

In step 336, candidate variants are identified from the filtered sequence reads. Since the RNA sequence reads are converted to DNA sequence reads, the human genome assembly GRCh37 (hg19) from Genome Reference Consortium may be used as a DNA reference genome for identifying the candidate variants. The Spliced Transcripts Alignment to a Reference (STAR) software may also be used for alignment of RNA. In step 338, quality scores are determined for each of the candidate variants. The processing system 200 may use a trained model 225 to determine the quality scores, which each indicate a likelihood that the candidate variant is a false positive detection of a mutation. The model 225 may be trained using data from processing calibration samples to determine an expected level of noise in samples of RNA molecules, e.g., by position. Once trained, the model 225 may predict whether a certain variant corresponds to noise that is typically observed in a sample of RNA molecules or a potential novel somatic mutation. The model 225 is further described below in Sections IV. Example Noise Models and V. Example Process Flows for Noise Models. In step 340, the processing system 200 outputs candidate variants having at least a threshold quality score. In other embodiments, the processing system 200 outputs candidate variants having less than or equal to a threshold quality score. The threshold quality score may be adjusted using information from calibration of RNA samples. For example, the processing system 200 or a trained model 225 takes into account data from a calibration curve of observed quality scores and theoretical quality scores from simulation, which is further described below with respect to FIG. 12. The output candidate variants may be used to determine predictions of cancer or disease diagnostics of an individual from whom the test sample was obtained, or of other individuals. In particular, mutations such as SNVs and indels associated with a type of cancer (or disease) are present in cancer cells. A cancer cell may express more copies of RNA transcripts with the cancer mutations than can be detected in a cfDNA sample of the individual. In some embodiments, the processing system 200 may be able to detect certain mutations with greater sensitivity using RNA molecules instead of DNA or cfDNA molecules. In an embodiment, sequence data from RNA samples and DNA or cfDNA samples may be combined as training data for a model.

In some embodiments, the processing system 200 outputs information about candidate variants by processing sequence reads from samples of one or both of DNA and RNA to be used as features (herein referred to as small variant features for clarity) in a machine learned model. These may include, but are not limited to a total number of somatic variants, a total number of nonsynonymous variants, a total number of synonymous variants, a presence or absence of somatic variants per gene, a presence or absence of somatic variants for particular genes that are known to be associated with at least one type of cancer, an allele frequency of a somatic variant per gene, order statistics according to AF of somatic variants, sensitivity or depth information from RNA sample calibration (further described below in Section VI. Example Detection of Variants in RNA), tumor or total mutational burden, and classification of somatic variants that are known to be associated with at least one type of cancer based on their allele frequency.

Small variant features may be used as input to one or more types of models such as a predictive cancer model. The predictive cancer model may generate predictions associated with cancer, e.g., predicting a likelihood that a given individual has, or is likely to develop, at least one type of cancer or disease. The predictive cancer model may be used to predict detections of one or more of stage I, stage II, stage III, and stage IV cancer. Example types of cancer include breast cancer, lung cancer, colorectal cancer, ovarian cancer, uterine cancer, melanoma, renal cancer, pancreatic cancer, thyroid cancer, gastric cancer, hepatobiliary cancer, esophageal cancer, prostate cancer, lymphoma, multiple myeloma, head and neck cancer, bladder cancer, cervical cancer, or any combination thereof. In some embodiments, the predictive cancer model is used to classify breast cancer as HR positive, HER2 overexpression, HER2 amplified, or triple negative, based on analysis of the sequence reads from a test sample. In other embodiments, small variant features may be used as input to one or more models as a predictive model for other diseases. For example, small variant features may be used in a predictive model for liver disease, cardiovascular disease, or other disease indications.

Predictive cancer models can be differently structured based on the particular features of the predictive cancer model. For example, the predictive cancer model can include one, two, three, or four different types of features, such as small variant features, whole genome features, methylation features, and baseline features. The predictive cancer model may also include one or more sub-models that use any combination of the aforementioned features. For example, in some embodiments, the model may use whole genome features derived from sequence reads that were generated by a whole genome sequencing assay (e.g., presence or absence of one or more copy number aberration). In some embodiments, the model may use one or more methylation feature derived from sequence reads that were generated using a methylation-aware assay, such as a whole genome bisulfite or targeted bisulfite sequencing assay. In still other embodiments, the model may use one or more baseline features. Baseline features refer to clinical symptoms or patient information.

FIG. 3C is flowchart of a method for generating RNA calibration data according to one embodiment. In step 352, a calibration sample including control RNA molecules and RNA molecules of an individual is obtained. The control RNA molecules may be a set of known RNA molecules provided by the External RNA Controls Consortium (ERCC), which may be referred to as ERCC Spike-In. The control RNA molecules do not include mutations (e.g., obtained from healthy individuals) and may be used as an experimental control to determine a baseline measurement of mutations in a sample of RNA molecules, e.g., with an unknown amount of mutations. The calibration sample may include a mixture of a predetermined ratio of RNA molecules of the individual to control RNA molecules, for example, 100:1, 500:1, 1000:1, etc.

In step 354, the processing system 200 determines sensitivity of detecting candidate variants in the calibration sample. The sensitivity may indicate a false-positive rate of mutations in the calibration sample. In step 356, the processing system 200 generates a mapping of depth of the calibration sample to the sensitivity. In some embodiments, the mapping may indicate an expected false-positive rate of mutations based on depth of one or more other types of sample attributes. In step 358, the processing system 200 determines candidate variants of the RNA molecules using the mapping. For example, referring to step 340 of the method 330 shown in FIG. 3B, the processing system 200 may determine or modify a value of the threshold quality score according to a performed calibration and resulting mapping. The mapping may be based on the calibration sample from step 352, or based on another previously processed calibration sample. In an embodiment, responsive to determining that sample has a greater sensitivity for mutations, the processing system 200 may determine a greater threshold quality score (e.g., applying a stricter filter) in comparison to a threshold quality score for another sample having lower sensitivity. As result of a greater threshold quality score, a fewer candidate variants will meet the threshold quality score, and thus the processing system 200 may call fewer candidate variants, relative to output using a lower threshold quality score.

FIG. 3D is flowchart of a method 360 for determining candidate variants based on sensitivity and depth of RNA sequence reads according to one embodiment. In step 362, calibration samples including one or more control RNA molecules and a RNA molecule of an individual is obtained. The calibration samples include molecules at different depths. The depth of the control RNA molecules or the RNA molecules of the individual may vary by position due to, for instance, greater expression at certain sections of the genome. In step 364, the processing system 200 generates mappings of the calibration samples. The sensitivity of a calibration sample may be based on the depth of the calibration sample. For instance, a detection rate of SNVs in the calibration samples may increase as the depth increases. Each of the mappings indicate a likelihood of detecting false positive mutations in the RNA molecule associated with the depth of a corresponding calibration sample. In other embodiments, the calibration samples may be mapped or organized using attributes other than depth. As examples, the calibration samples including RNA molecules may vary in AF, or may be associated with different types of cancers or different stages of cancer.

In step 366, the processing system 200 determines a depth of the RNA molecule at a certain position. In step 368, the processing system 200 determines candidate variants of the RNA molecule using the mappings and the depth. For instance, the processing system 200 uses the depth to lookup a corresponding sensitivity of SNVs in the mappings.

### IV. Example Noise Models

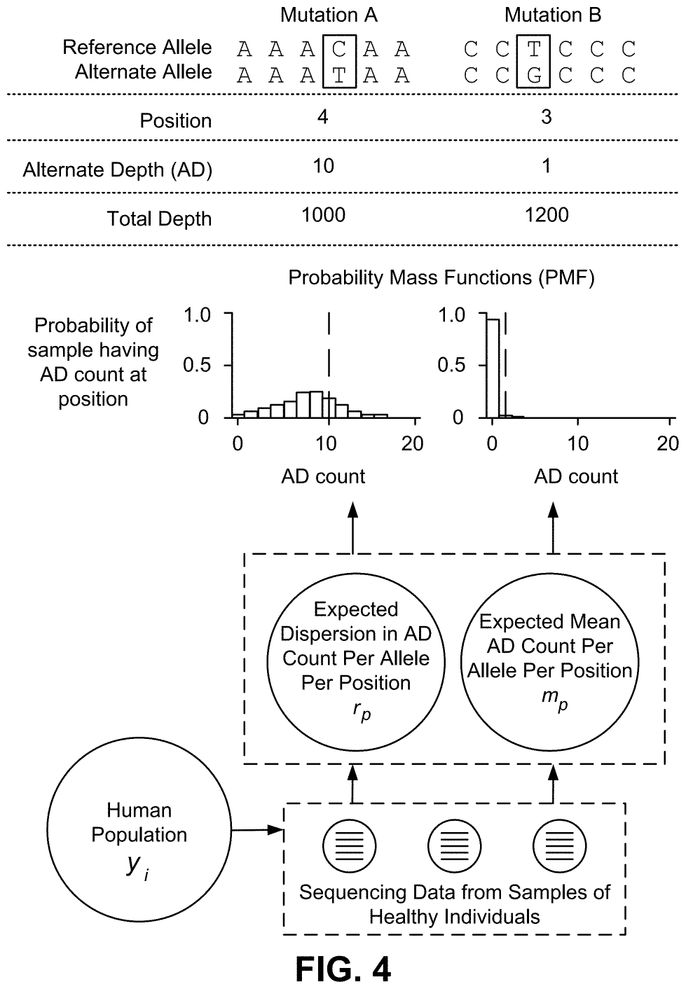

FIG. 4 is a diagram of an application of a Bayesian hierarchical model 225 according to one embodiment. Mutation A and Mutation B are shown as examples for purposes of explanation. The following processes using the Bayesian hierarchical model 225 may be applied to candidate variants determined or generated in the previously described methods of FIGS. 3A-C. In the embodiment of FIG. 4, Mutations A and B are represented as SNVs, though in other embodiments, the following description is also applicable to indels or other types of mutations. Mutation A is a C>T mutation at position 4 of a first reference allele from a first sample. The first sample has a first AD of 10 and a first total depth of 1000. Mutation B is a T>G mutation at position 3 of a second reference allele from a second sample. The second sample has a second AD of 1 and a second total depth of 1200. Based merely on AD (or AF), Mutation A may appear to be a true positive, while Mutation B may appear to be a false positive because the AD (or AF) of the former is greater than that of the latter. However, Mutations A and B may have different relative levels of noise rates per allele and/or per position of the allele. In fact, Mutation A may be a false positive and Mutation B may be a true positive, once the relative noise levels of these different positions are accounted for. The models 225 described herein model this noise for appropriate identification of true positives accordingly.

The probability mass functions (PMFs) illustrated in FIG. 4 indicate the probability (or likelihood) of a sample from a subject having a given AD count at a position. Using sequencing data from samples of healthy individuals (e.g., stored in the sequence database 210), the processing system 200 trains a model 225 from which the PDFs for healthy samples may be derived. In particular, the PDFs are based on mₚ, which models the expected mean AD count per allele per position in normal tissue (e.g., of a healthy individual), and rₚ, which models the expected variation (e.g., dispersion) in this AD count. Stated differently, mₚ and/or rₚ represent a baseline level of noise, on a per position per allele basis, in the sequencing data for normal tissue.

Using the example of FIG. 4 to further illustrate, samples from the healthy individuals represent a subset of the human population modeled by yᵢ, where i is the index of the healthy individual in the training set. Assuming for sake of example the model 225 has already been trained, PDFs produced by the model 225 visually illustrate the likelihood of the measured ADs for each mutation, and therefore provide an indication of which are true positives and which are false positives. The example PDF on the left of FIG. 4 associated with Mutation A indicates that the probability of the first sample having an AD count of 10 for the mutation at position 4 is approximately 20%. Additionally, the example PDF on the right associated with Mutation B indicates that the probability of the second sample having an AD count of 1 for the mutation at position 3 is approximately 1% (note: the PDFs of FIG. 4 are not exactly to scale). Thus, the noise rates corresponding to these probabilities of the PDFs indicate that Mutation A is more likely to occur than Mutation B, despite Mutation B having a lower AD and AF. Thus, in this example, Mutation B may be the true positive and Mutation A may be the false positive. Accordingly, the processing system 200 may perform improved variant calling by using the model 225 to distinguish true positives from false positives at a more accurate rate, and further provide numerical confidence as to these likelihoods.

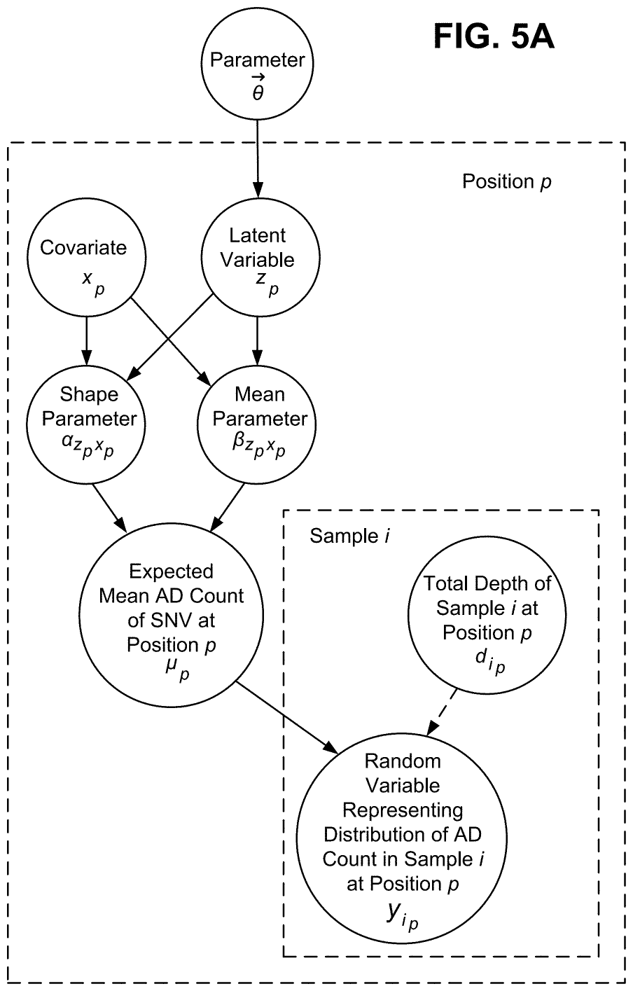

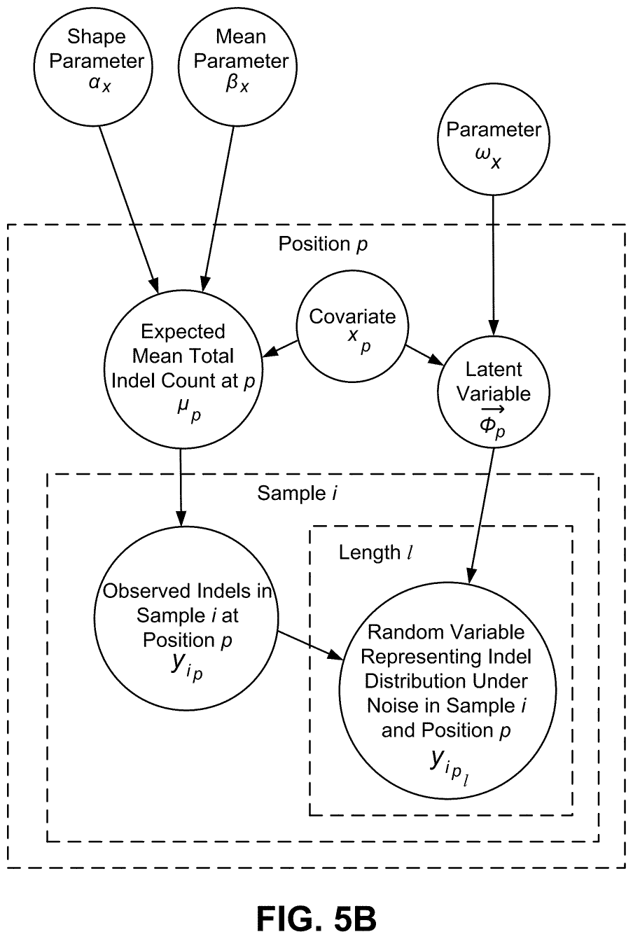

FIG. 5A shows dependencies between parameters and sub-models of a Bayesian hierarchical model 225 for determining true single nucleotide variants according to one embodiment. Parameters of models may be stored in the parameter database 230. In the example shown in FIG. 5A, \vec{θ} represents the vector of weights assigned to each mixture component. The vector \vec{θ} takes on values within the simplex in K dimensions and may be learned or updated via posterior sampling during training. It may be given a uniform prior on said simplex for such training. The mixture component to which a position p belongs may be modeled by latent variable zₚ using one or more different multinomial distributions:

zₚ˜Multinom(\vec{θ})

Together, the latent variable zₚ, the vector of mixture components \vec{θ}, α, and β allow the model for μ, that is, a sub-model of the Bayesian hierarchical model 225, to have parameters that “pool” knowledge about noise, that is they represent similarity in noise characteristics across multiple positions. Thus, positions of sequence reads may be pooled or grouped into latent classes by the model. Also advantageously, samples of any of these “pooled” positions can help train these shared parameters. A benefit of this is that the processing system 200 may determine a model of noise in healthy samples even if there is little to no direct evidence of alternative alleles having been observed for a given position previously (e.g., in the healthy tissue samples used to train the model).

The covariate xₚ (e.g., a predictor) encodes known contextual information regarding position p which may include, but is not limited to, information such as trinucleotide context, mappability, segmental duplication, or other information associated with sequence reads. Trinucleotide context may be based on a reference allele and may be assigned numerical (e.g., integer) representation. For instance, “AAA” is assigned 1, “ACA” is assigned 2, “AGA” is assigned 3, etc. Mappability represents a level of uniqueness of alignment of a read to a particular target region of a genome. For example, mappability is calculated as the inverse of the number of position(s) where the sequence read will uniquely map. Segmental duplications correspond to long nucleic acid sequences (e.g., having a length greater than approximately 1000 base pairs) that are nearly identical (e.g., greater than 90% match) and occur in multiple locations in a genome as result of natural duplication events (e.g., not associated with a cancer or disease).

The expected mean AD count of a SNV at position p is modeled by the parameter μₚ. For sake of clarity in this description, the terms μₚ and yₚ refer to the position specific sub-models of the Bayesian hierarchical model 225. In one embodiment, μₚ is modeled as a Gamma-distributed random variable having shape parameter α_{z}_{ₚ}_{,x}_{ₚ} and mean parameter β_{z}_{ₚ}_{,x}_{ₚ}:

μₚ˜Gamma(α_{z}_{ₚ}_{,x}_{ₚ}, β_{z}_{ₚ}_{,x}_{ₚ})

In other embodiments, other functions may be used to represent μₚ, examples of which include but are not limited to: a log-normal distribution with log-mean y_{z}_{ₚ} and log-standard-deviation τ_{z}_{ₚ}ₓ_{ₚ}, a Weibull distribution, a power law, an exponentially-modulated power law, or a mixture of the preceding.

In the example shown in FIG. 5A, the shape and mean parameters are each dependent on the covariate xₚ and the latent variable zₚ, though in other embodiments, the dependencies may be different based on various degrees of information pooling during training. For instance, the model may alternately be structured so that α_{z}_{ₚ} pdepends on the latent variable but not the covariate. The distribution of AD count of the SNV at position p in a human population sample i (of a healthy individual) is modeled by the random variable yᵢ_{ₚ}. In one embodiment, the distribution is a Poisson distribution given a depth dᵢₚ of the sample at the position:

yᵢ_{ₚ}|dᵢ_{ₚ}˜Poisson(dᵢ_{ₚ}·μₚ)

In other embodiments, other functions may be used to represent yᵢ_{ₚ}, examples of which include but are not limited to: negative binomial, Conway-Maxwell-Poisson distribution, zeta distribution, and zero-inflated Poisson.

FIG. 5B shows dependencies between parameters and sub-models of a Bayesian hierarchical model for determining true insertions or deletions according to one embodiment. In contrast to the SNV model shown in FIG. 5A, the model for indels shown in FIG. 5B includes different levels of hierarchy. The covariate xₚ encodes known features at position p and may include, e.g., a distance to a homopolymer, distance to a RepeatMasker repeat, or other information associated with previously observed sequence reads. Latent variable \vec{φₚ} may be modeled by a Dirichlet distribution based on parameters of vector \vec{ω}ₓ, which represent indel length distributions at a position and may be based on the covariate. In some embodiments, \vec{ω}ₓ is also shared across positions (\vec{ω}_{x,p}) that share the same covariate value(s). Thus for example, the latent variable may represent information such as that homopolymer indels occur at positions 1, 2, 3, etc. base pairs from the anchor position, while trinucleotide indels occur at positions 3, 6, 9, etc. from the anchor position.

The expected mean total indel count at positionp is modeled by the distribution μₚ. In some embodiments, the distribution is based on the covariate and has a Gamma distribution having shape parameter αₓ_{ₚ} and mean parameter βₓ_{ₚ}_{z}_{ₚ}:

μₚ˜Gamma(α_{z}_{ₚ}, βₓ_{ₚ}_{z}_{ₚ})

In other embodiments, other functions may be used to represent μₚ, examples of which include but are not limited to: negative binomial, Conway-Maxwell-Poisson distribution, zeta distribution, and zero-inflated Poisson.

The observed indels at position p in a human population sample i (of a healthy individual) is modeled by the distribution yᵢ_{ₚ}. Similar to example in FIG. 5A, in some embodiments, the distribution of indel intensity is a Poisson distribution given a depth dᵢ_{ₚ} of the sample at the position:

yᵢ_{ₚ}|dᵢ_{ₚ}˜Poisson(diᵢ_{ₚ}·μₚ)

In other embodiments, other functions may be used to represent yᵢ_{ₚ}, examples of which include but are not limited to: negative binomial, Conway-Maxwell-Poisson distribution, zeta distribution, and zero-inflated Poisson.

Due to the fact that indels may be of varying lengths, an additional length parameter is present in the indel model that is not present in the model for SNVs. As a consequence, the example model shown in FIG. 5B has an additional hierarchical level (e.g., another sub-model), which is again not present in the SNV models discussed above. The observed count of indels of length l (e.g., up to 100 or more base pairs of insertion or deletion) at positionp in sample i is modeled by the random variable yᵢ_{ₚₗ}, which represents the indel distribution under noise conditional on parameters. The distribution may be a multinomial given indel intensity yᵢ_{ₚ} of the sample and the distribution of indel lengths \vec{φₚ} at the position:

yᵢ_{ₚₗ}|yᵢ_{ₚ}, \vec{φₚ}˜Multinom(yᵢ_{ₚ}, \vec{φₚ})

In other embodiments, a Dirichlet-Multinomial function or other types of models may be used to represent yᵢ_{ₚₗ}.

By architecting the model in this manner, the machine learning engine 220 may decouple learning of indel intensity (i.e., noise rate) from learning of indel length distribution. Independently determining inferences for an expectation for whether an indel will occur in healthy samples and expectation for the length of the indel at a position may improve the sensitivity of the model. For example, the length distribution may be more stable relative to the indel intensity at a number of positions or regions in the genome, or vice versa.

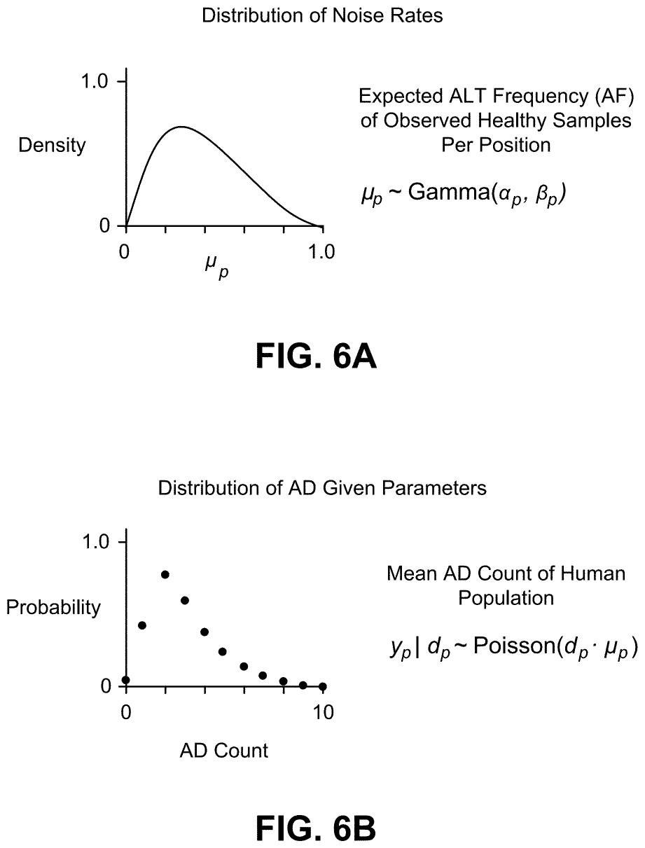

FIGS. 6A-B illustrate diagrams associated with a Bayesian hierarchical model 225 according to one embodiment. The graph shown in FIG. 6A depicts the distribution μₚ of noise rates, i.e., likelihood (or intensity) of SNVs or indels for a given position as characterized by a model. The continuous distribution represents the expected AF μₚ of non-cancer or non-disease mutations (e.g., mutations naturally occurring in healthy tissue) based on training data of observed healthy samples from healthy individuals (e.g., retrieved from the sequence database 210). Though not shown in FIG. 6A, in some embodiments, the shape and mean parameters of μₚ may be based on other variables such as the covariate xₚ or latent variable zₚ. The graph shown in FIG. 6B depicts the distribution of AD at a given position for a sample of a subject, given parameters of the sample such as sequencing depth dₚ at the given position. The discrete probabilities for a draw of μₚ are determined based on the predicted true mean AD count of the human population based on the expected mean distribution μₚ.

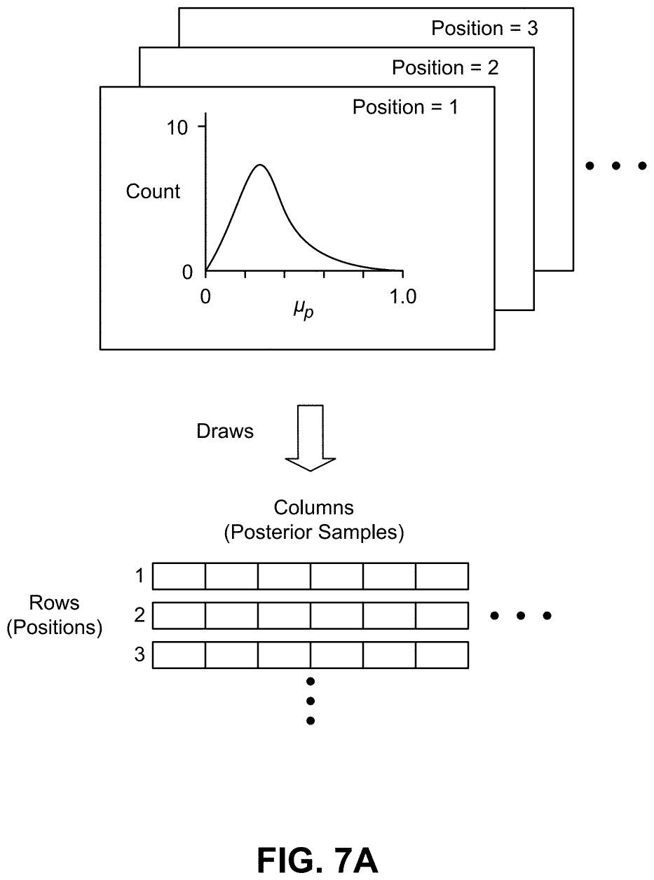

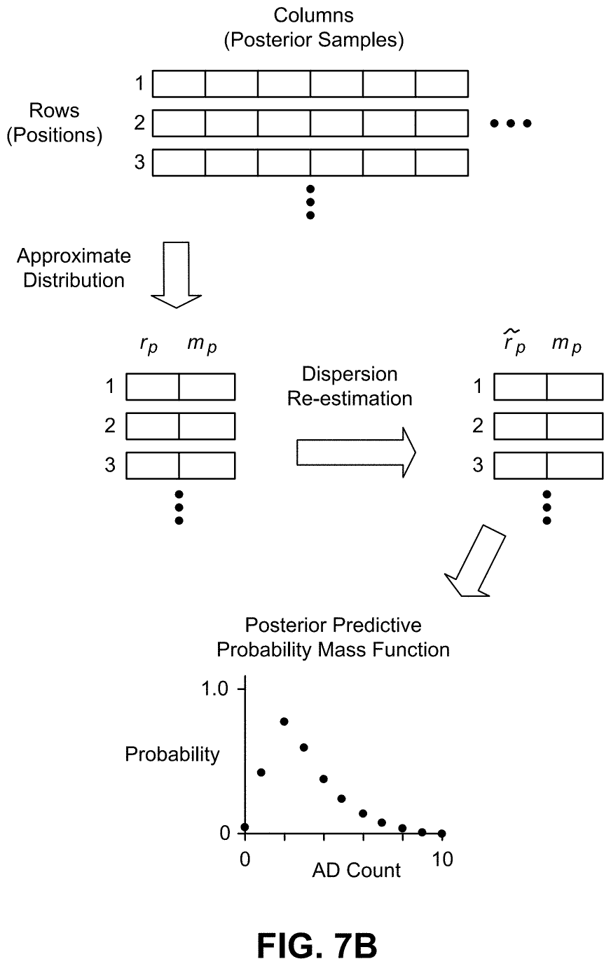

FIG. 7A is a diagram of an example process for determining parameters by fitting a Bayesian hierarchical model 225 according to one embodiment. To train a model, the machine learning engine 220 samples iteratively from a posterior distribution of expected noise rates (e.g., the graph shown in FIG. 6B) for each position of a set of positions. The machine learning engine 220 may use Markov chain Monte Carlo (MCMC) methods for sampling, e.g., a Metropolis-Hastings (MH) algorithm, custom MH algorithms, Gibbs sampling algorithm, Hamiltonian mechanics-based sampling, random sampling, among other sampling algorithms. During Bayesian Inference training, parameters are drawn from the joint posterior distribution to iteratively update all (or some) parameters and latent variables of the model (e.g., \vec{θ}, zₚ, α_{z}_{ₚ}_{,x}_{ₚ}, β_{z}_{ₚ}_{, x}_{ₚ}, μₚ, etc.).

In one embodiment, the machine learning engine 220 performs model fitting by storing draws of μₚ, the expected mean counts of AF per position and per sample, in the parameters database 230. The model is trained or fitted through posterior sampling, as previously described. In an embodiment, the draws of μₚ are stored in a matrix data structure having a row per position of the set of positions sampled and a column per draw from the joint posterior (e.g., of all parameters conditional on the observed data). The number of rows R may be greater than 6 million and the number of columns for N iterations of samples may be in the thousands. In other embodiments, the row and column designations are different than the embodiment shown in FIG. 7A, e.g., each row represents a draw from a posterior sample, and each column represents a sampled position (e.g., transpose of the matrix example shown in FIG. 7A).

FIG. 7B is a diagram of using parameters from a Bayesian hierarchical model 225 to determine a likelihood of a false positive according to one embodiment. The machine learning engine 220 may reduce the R rows-by-N column matrix shown in FIG. 7A into an R rows-by-2 column matrix illustrated in FIG. 7B. In one embodiment, the machine learning engine 220 determines a dispersion parameter rₚ (e.g., shape parameter) and mean parameter mₚ (which may also be referred to as a mean rate parameter mₚ) per position across the posterior samples μₚ. The dispersion parameter rₚ may be determined as

$${r}_{p} = \frac{{m}_{p}^{2}}{{v}_{p}^{2}} ,$$

where mₚ and vₚ are the mean and variance of the sampled values of μₚ at the position, respectively. Those of skill in the art will appreciate that other functions for determining rₚ may also be used such as a maximum likelihood estimate.

The machine learning engine 220 may also perform dispersion re-estimation of the dispersion parameters in the reduced matrix, given the mean parameters. In one embodiment, following Bayesian training and posterior approximation, the machine learning engine 220 performs dispersion re-estimation by retraining for the dispersion parameters [symbol] based on a negative binomial maximum likelihood estimator per position. The mean parameter may remain fixed during retraining. In one embodiment, the machine learning engine 220 determines the dispersion parameters r′ₚ at each position for the original AD counts of the training data (e.g., yᵢ_{ₚ} and dᵢ_{ₚ} based on healthy samples). The machine learning engine 220 determines [symbol] =max(rₚ, r′ₚ), and stores [symbol] in the reduced matrix. Those of skill in the art will appreciate that other functions for determining [symbol] may also be used, such as a method of moments estimator, posterior mean, or posterior mode.

During application of trained models, the processing system 200 may access the dispersion (e.g., shape) parameters [symbol] and mean parameters mₚ to determine a function parameterized by [symbol] and mₚ. The function may be used to determine a posterior predictive probability mass function (or probability density function) for a new sample of a subject. Based on the predicted probability of a certain AD count at a given position, the processing system 200 may account for site-specific noise rates per position of sequence reads when detecting true positives from samples. Referring back to the example use case described with respect to FIG. 4, the PDFs shown for Mutations A and B may be determined using the parameters from the reduced matrix of FIG. 7B. The posterior predictive probability mass functions may be used to determine the probability of samples for Mutations A or B having an AD count at certain position.

### V. Example Process Flows for Noise Models

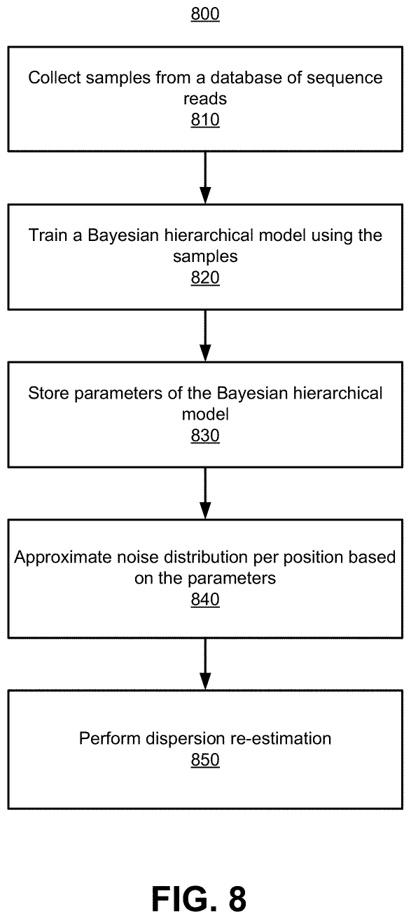

FIG. 8 is flowchart of a method 800 for training a Bayesian hierarchical model 225 according to one embodiment. In step 810, the machine learning engine 220 collects samples, e.g., training data, from a database of sequence reads (e.g., the sequence database 210). In step 820, the machine learning engine 220 trains the Bayesian hierarchical model 225 using the samples using a Markov Chain Monte Carlo method. During training, the model 225 may keep or reject sequence reads conditional on the training data. The machine learning engine 220 may exclude sequence reads of healthy individuals that have less than a threshold depth value or that have an AF greater than a threshold frequency in order to remove suspected germline mutations that are not indicative of target noise in sequence reads. In other embodiments, the machine learning engine 220 may determine which positions are likely to contain germline variants and selectively exclude such positions using thresholds like the above. In one embodiment, the machine learning engine 220 may identify such positions as having a small mean absolute deviation of AFs from germline frequencies (e.g., 0, ½, and 1).

The Bayesian hierarchical model 225 may update parameters simultaneously for multiple (or all) positions included in the model. Additionally, the model 225 may be trained to model expected noise for each ALT. For instance, a model for SNVs may perform a training process four or more times to update parameters (e.g., one-to-one substitutions) for mutations of each of the A, T, C, and G bases to each of the other three bases. In step 830, the machine learning engine 220 stores parameters of the Bayesian hierarchical model 225 (e.g., ensemble parameters output by the Markov Chain Monte Carlo method). In step 840, the machine learning engine 220 approximates noise distribution (e.g., represented by a dispersion parameter and a mean parameter) per position based on the parameters. In step 850, the machine learning engine 220 performs dispersion re-estimation (e.g., maximum likelihood estimation) using original AD counts from the samples (e.g., training data) used to train the Bayesian hierarchical model 225.

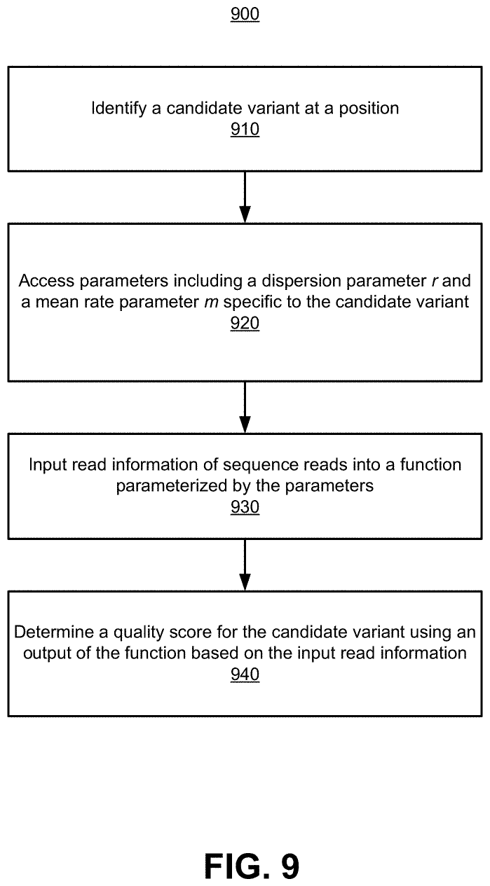

FIG. 9 is flowchart of a method 900 for determining a likelihood of a false positive according to one embodiment. At step 910, the processing system 200 identifies a candidate variant, e.g., at a position p of a sequence read, from a set of sequence reads, which may be sequenced from a cfRNA or cfDNA sample obtained from an individual. At step 920, the processing system 200 accesses parameters, e.g., dispersion and mean rate parameters [symbol] and mₚ, respectively, specific to the candidate variant, which may be based on the position p of the candidate variant. The parameters may be derived using a model, e.g., a Bayesian hierarchical model 225 representing a posterior predictive distribution with an observed depth of a given sequence read and a mean parameter μₚ at the position p as input. In an embodiment, the mean parameter μₚ is a gamma distribution encoding a noise level of nucleotide mutations with respect to the position p for a training sample.

At step 930, the processing system 200 inputs read information (e.g., AD or AF) of the set of sequence reads into a function (e.g., based on a negative binomial) parameterized by the parameters, e.g., [symbol] and mₚ. At step 940, the processing system 200 (e.g., the score engine 235) determines a quality score for the candidate variant (e.g., at the position p) using an output of the function based on the input read information. The quality score may indicate a likelihood of seeing an allele count for a given sample (e.g., from a subject) that is greater than or equal to a determined allele count of the candidate variant (e.g., determined by the model and output of the function). The processing system 200 may convert the likelihood into a Phred-scaled score. In some embodiments, the processing system 200 uses the likelihood to determine false positive mutations responsive to determining that the likelihood is less than a threshold value. In some embodiments, the processing system 200 uses the function to determine that a sample of sequence reads includes at least a threshold count of alleles corresponding to a gene found in sequence reads from a tumor biopsy of an individual. Responsive to this determination, the processing system 200 may predict presence of cancer cells in the individual based on variant calls. In some embodiments, the processing system 200 may perform weighting based on quality scores, use the candidate variants and quality scores for false discovery methods, annotate putative calls with quality scores, or provision to subsequent systems.

The processing system 200 may use functions encoding noise levels of nucleotide mutations with respect to a given training sample for downstream analysis. In some embodiments, the processing system 200 uses the aforementioned negative binomial function parameterized by the dispersion and mean rate parameters [symbol] and mₚ to determine expected noise for a particular nucleic acid position within a sample. Moreover, the processing system 200 may derive the parameters by training a Bayesian hierarchical model 225 using training data associated with the particular nucleic acid sample.

### VI. Example Detection of Variants in RNA

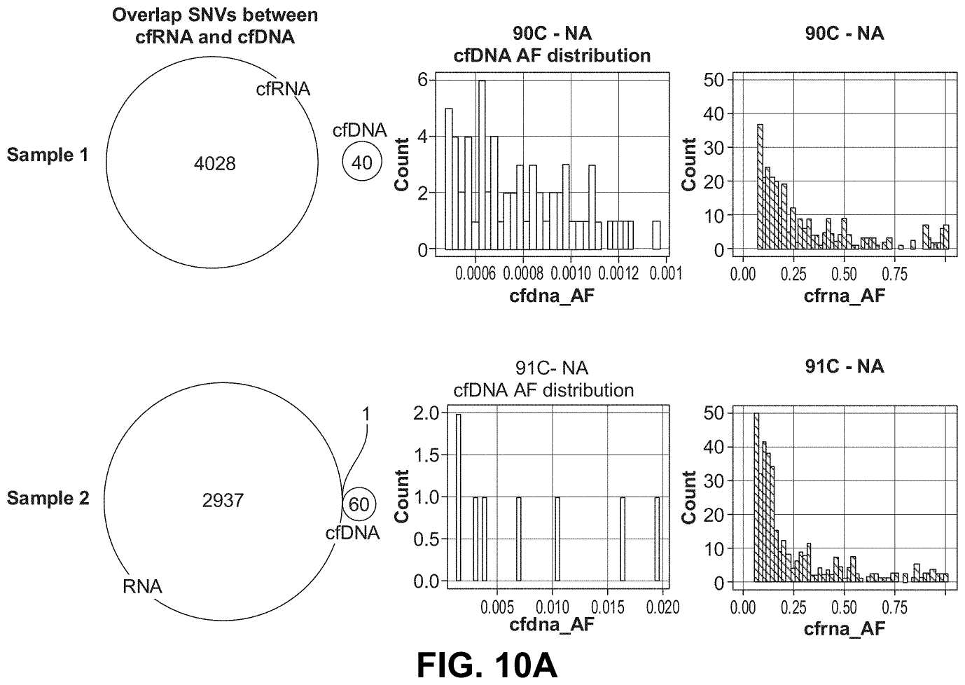

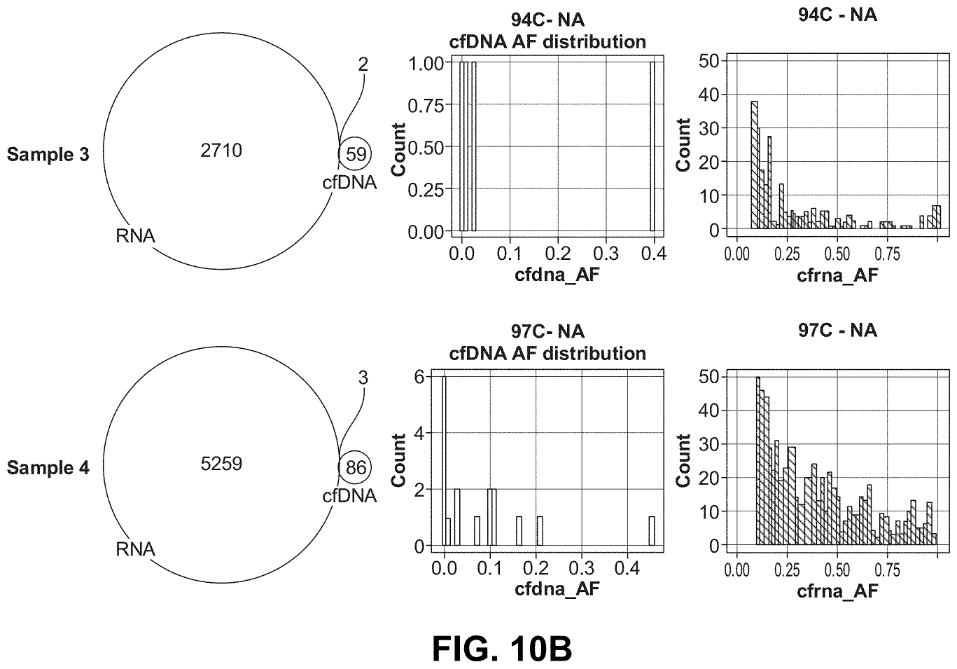

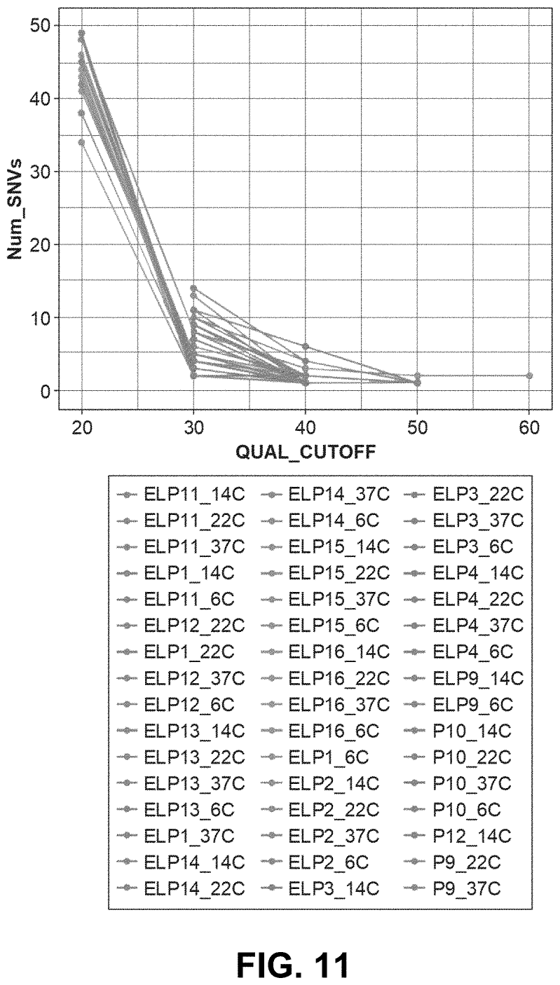

The data shown in FIGS. 10-18B were obtained by processing two different RNA samples. A first RNA sample was used to determine the data shown in FIGS. 10A-10B, 15, 16A-B, and 18A-B. A second RNA sample was used to determine the data shown in FIGS. 11, 12, 13, 14, and 17. The first RNA sample includes sequence reads of healthy individuals as well as individuals having prostate or lung cancer. Additionally, the first RNA sample does not include control RNA molecules. The second RNA sample includes sequence reads from blood samples drawn from healthy individuals and does not include cancer samples. The second RNA sample also includes ERCC Spike-In, i.e., control RNA molecules.

Both the first and second RNA samples were processed using an assay including previously described steps of the methods, e.g., as shown in FIGS. 1A and 3A-D. As example steps, the Qiagen QIAmp Circulating Nucleic Acid kit microRNA protocol was used to extract cfRNA from isolated blood plasma samples of the individuals. The sample was then treated with a DNase (Qiagen) to deplete abundant ribosomal RNA (rRNA) molecules. Reverse transcription was performed to synthesize complementary DNA (cDNA) molecules from the RNA molecules. Double-stranded cDNA molecules were generated using a reaction mixture comprising primers and a polymerase. A sequencing library was prepared from the double-stranded cDNA molecules by ligating adapters to both ends of the double-stranded cDNA molecules and amplifying the constructs. The sequencing library was enriched using GRAIL's proprietary pan-cancer 507-gene liquid biopsy panel and the enriched sample sequenced using a HiSeq X (Illumina, San Diego, Calif.).

FIGS. 10A and 10B are diagrams comparing the number of cfDNA and cfRNA and the alternate frequency (AF) of cfDNA and cfRNA variants detected without using a noise model. The SNVs detected in cfRNA and cfDNA are obtained from plasma samples of four individuals with non-small cell lung cancer. As illustrated across samples 1 through 4 of both cfRNA and cfDNA, the processing system 200 detects a greater number of false positive SNVs in the cfRNA than in the cfDNA. Additionally, there is a small overlap of SNVs between cfRNA and cfDNA for samples 2 through 4. The numbers of SNVs detected in cfRNA are on the scale of thousands, while the numbers of SNVs detected in cfDNA are on the scale of tens. Without filtering or using a model to account for expected levels of noise, there may be too many false-positive detections of SNVs in cfRNA when processing sequence reads to make accurate or useful predictions regarding cancer or disease based on SNVs.

FIG. 11 is a diagram showing the number of variants detected at various threshold quality scores in example samples according to one embodiment. The threshold quality scores may represent cutoff values for calling variants. Across the illustrated samples including different control RNA molecules, the number of false positive SNVs detected using a model 225 decreases as the threshold quality score increases. As indicated by the legend of the diagram, multiple types of control RNA molecule samples may be used to provide wider coverage of RNA transcripts corresponding to different regions of a transcriptome.

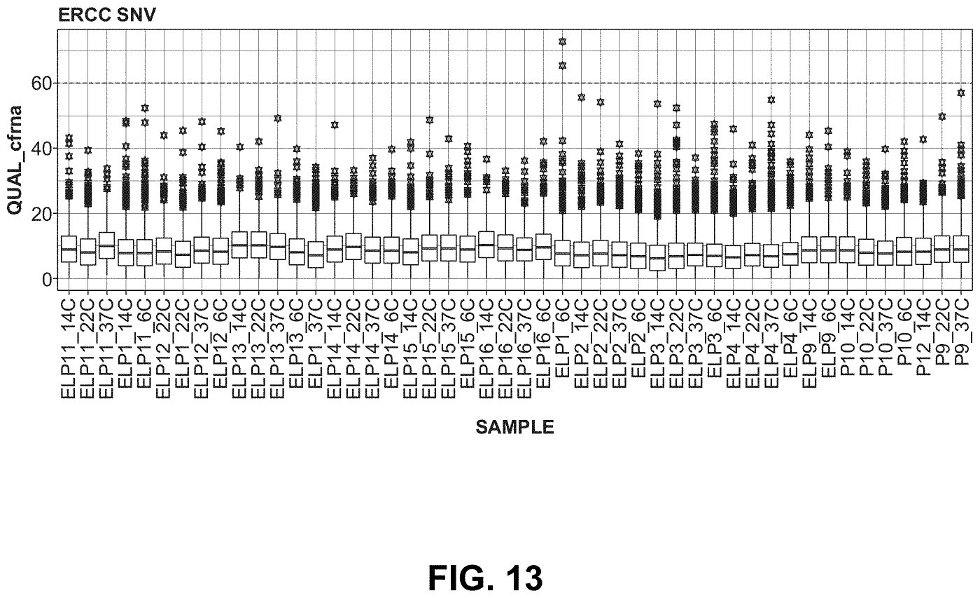

FIG. 12 is a diagram of observed and theoretical quality scores according to one embodiment. According to the Phred scale, the theoretical quality score QUAL_{theory}=−10·log₁₀ (prob of QUAL≥QUAL_{cutoff}) may be determined based on the log of probability that the true quality score of a candidate variant QUAL is greater than a given threshold quality score QUAL_{cutoff}. For a perfectly calibrated model, the theoretical quality score should equal the threshold quality score. Based on the simulated data using samples of control RNA molecules shown in the calibration curve of FIG. 12, the observed quality score determined by a model 225, e.g., a Bayesian hierarchical model 225 as previously described in Section IV. Example Noise Models, tends to be close though slightly greater than the corresponding expected quality score, represented by the dotted line. This illustrated trend indicates that the model 225 may be determining conservative estimates of quality scores in simulation, and thus may miss calling some true positives. The processing system 200 or model 225 may use the calibration curve to adjust a threshold quality score for calling candidate variants as illustrated in FIG. 13 described below. In some embodiments, the processing system 200 maps the raw quality score from the x-axis “QUAL_cutoff” to the corresponding adjusted quality score from the y-axis “QUAL_observed.”

FIG. 13 is a diagram showing the of distribution of quality scores for calibration samples of RNA according to one embodiment. The dotted line shown in the diagram indicates a threshold quality score of 60, which corresponds to a false detection rate (FDR) of 10′ based on the Phred scale. A model 225 determines the theoretical expected number of false-positive SNVs detected from a sample of control RNA molecules based on the sequence size (e.g., number of nucleotide base pairs in a fragment) and false detection rate (e.g., false positives per mega-base pairs):

false positives=size*false detection rate

For an example control RNA molecule sample size of 20,460 sequence reads and a FDR of 10′, the number of false positives expected is 0.02046. The distribution shown in FIG. 11 includes two false positive SNVs detected by a Bayesian hierarchical model 225, where the false positives have quality scores greater than the quality score cutoff of 60.

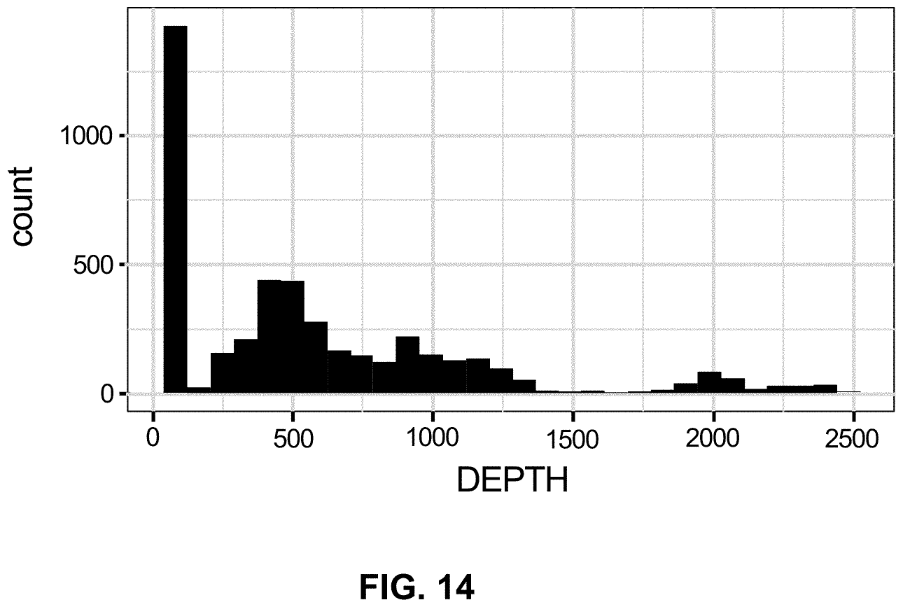

FIG. 14 is a diagram showing depth distributions observed for control RNA molecules according to one embodiment. The samples of control RNA molecules may have different concentrations based on varying transcript levels. Control RNA molecules (e.g., ERCC) received from a third party vendor may be diluted to reflect expected levels of sequencing depth (e.g., coverage of transcripts) over particular loci of a genome from real samples. Generally, RNA transcripts at certain loci may be expressed at a greater magnitude than transcripts at other loci. The depth information from calibration may be used to adjust a threshold quality score for variant calling of test samples. In the embodiment of FIG. 14, the distribution determined by a model 225 indicates peaks around depths of approximately 100, 300-700, and 1500-2500. Regions of the genome corresponding to depths of 300-700 may be used to perform more accurate variant calling because the signal in regions corresponding to depths of 100 or 1500-2500 may be too low or high. In other embodiments, the expected or observed distribution of counts based on sequencing depth may exhibit different ranges of peaks or trends.

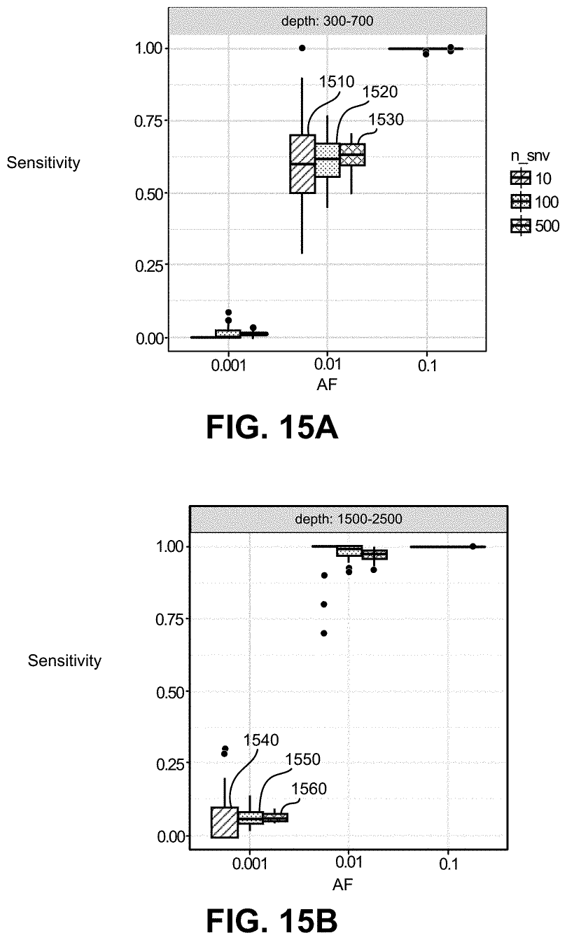

FIGS. 15A and 15B are diagrams of sensitivity based on depth and alternate frequency of calibration samples of RNA according to various embodiments. The diagram shows data from simulation at allele frequencies of 0.001, 0.01, and 0.1, as well as at SNVs counts of 10, 100, and 500. FIG. 15A includes boxplot 1510 representing samples in which at least 10 SNVs were detected, boxplot 1520 representing samples in which at least 100 SNVs were detected, and boxplot 1530 representing samples in which at least 500 SNVs were detected. FIG. 15B includes boxplot 1540 representing samples in which at least 10 SNVs were detected, boxplot 1550 representing samples in which at least 100 SNVs were detected, and boxplot 1560 representing samples in which at least 500 SNVs were detected.

As illustrated by the box plots in FIGS. 15A and 15B of samples having depths in the ranges of 300-700 and 1500-2000, respectively, sensitivity of a Bayesian hierarchical model 225 generally increases as the depth increases. In particular, at an AF of 0.01, the median sensitivity corresponding to depth 300-700 is between 0.50 and 0.75, while the median sensitivity corresponding to depth 1500-2500 is close to 1.00. The box plots also show that the sensitivity of the model 225 generally increases as the AF increases, e.g., because there is greater coverage of the control RNA molecules. The sensitivity can be used to adjust a threshold quality score of the model 225 for variant calling. In addition, the processing system 200 may generate mappings between depths and corresponding sensitivities. The mappings may be used in any number of downstream processes, for example, as a weight or feature for a (e.g., small variant) classifier. In particular, the processing system 200 may use data from calibration such as sensitivities or adjusted quality scores to filter false positives from RNA samples, which may result in more accurate features for a classifier or model training. In some embodiments, the processing system 200 uses mapping or sensitivity data to determine a confidence interval for predictions of allele frequency of a sample.

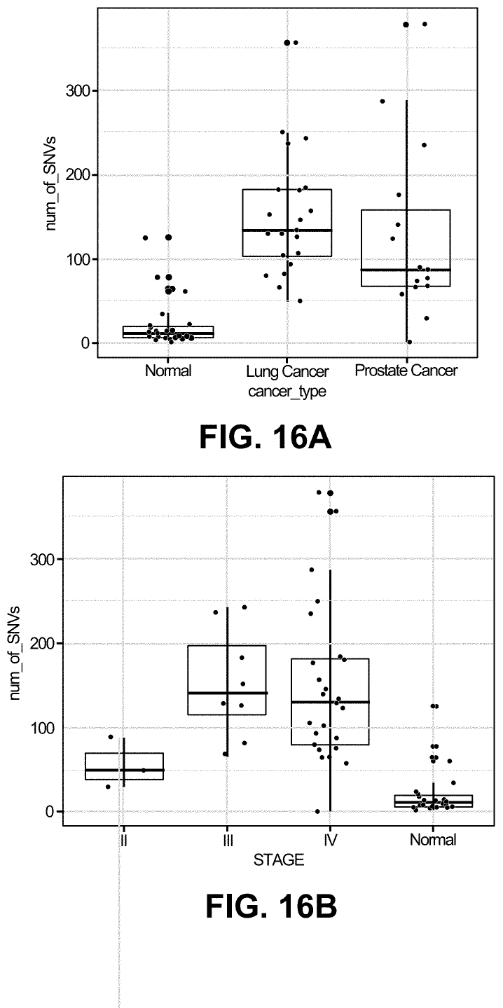

FIGS. 16A and 16B are diagrams showing the number of variants detected in cfRNA samples based on type and stage of cancer according to various embodiments. Particularly, FIG. 16A shows counts of SNVs detected using a Bayesian hierarchical model 225 for RNA noise estimation for normal samples, e.g., from healthy individuals, as well as samples from individuals known to have lung or prostate cancer. FIG. 16B shows counts of SNVs detected for normal samples and samples associated with stage II, III, and IV cancer. The median and variance of SNVs detected in normal samples are both less than those statistics for SNVs detected in samples from individuals known to have some cancer. Furthermore, as shown by the box plots, the processing system 200 detects variants (SNVs) on the scale of tens to hundreds, in comparison to the scale of thousands of false positive variants detected without using the model 225 as previously shown in FIGS. 10A-10B. By distinguishing between healthy and cancer samples and reducing the number of false positive detections of variants in RNA, the model 225 may provide improved sensitivity of variant calling using RNA molecules, which may be used downstream for cancer or disease detection.

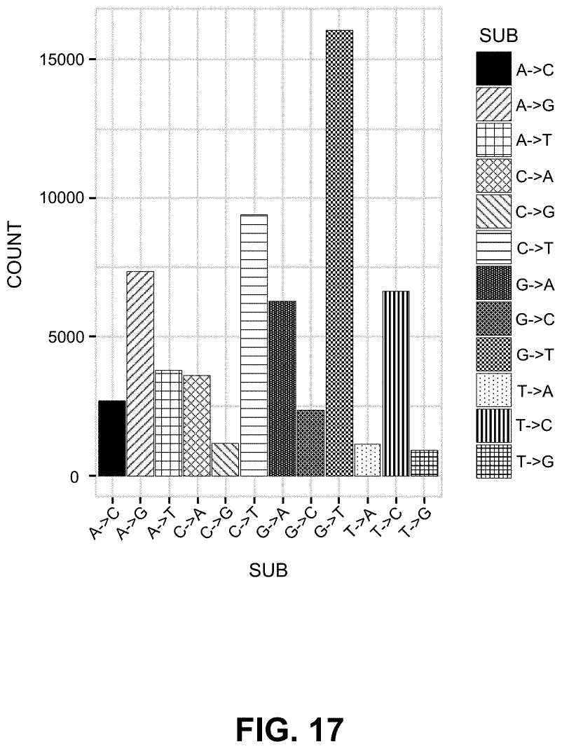

FIG. 17 is a diagram showing different types of variants detected in cfRNA samples according to one embodiment. As shown in the diagram, the G->T substitution mutation had the greatest prevalence among the types of SNVs detected by a model 225. This may be at least partly due to oxidative damages that are also prevalent in cfDNA. Other possible sources of variants, or variation in counts of the different types of variants, include artifacts or noise from sample processing and collapsing sequence reads.

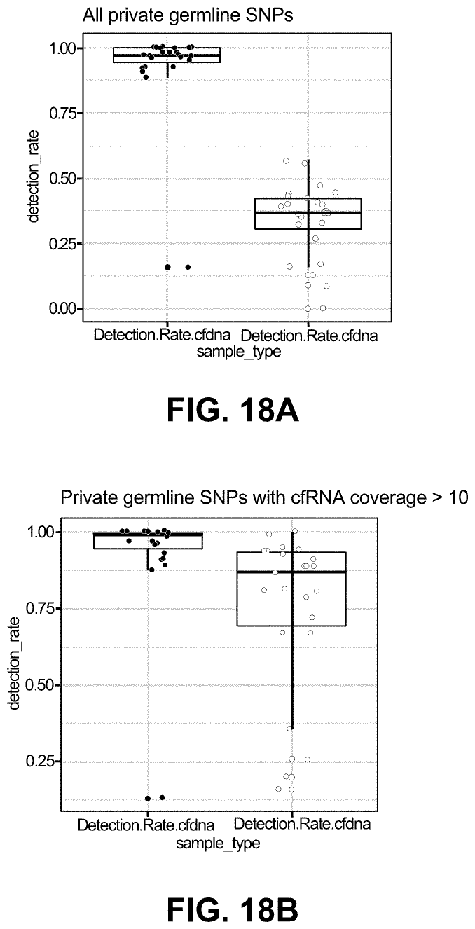

FIGS. 18A and 18B are diagrams showing comparisons of variant detection rates in cfDNA and cfRNA samples according to various embodiments. The model 225 used to determine the illustrated detection rates used a threshold quality score of 60 (on the Phred scale) and was trained using leave-one-out cross validation. As illustrated by the box plot in FIG. 18B, the detection rate determined by the model 225 for cfRNA variants having coverage greater than ten is similar to the detection rate determined for cfDNA variants. Furthermore, the detection rate determined using the model 225 for cfRNA variants is greater for samples with cfRNA coverage greater than ten than for all private germline samples. This difference is less apparent when comparing cfDNA variant detection rates between the two coverage conditions. Due to natural variations in coverage of RNA transcripts at different loci, some portions germline samples with detected SNVs have lower depth.

### VII. Computing Machine Architecture

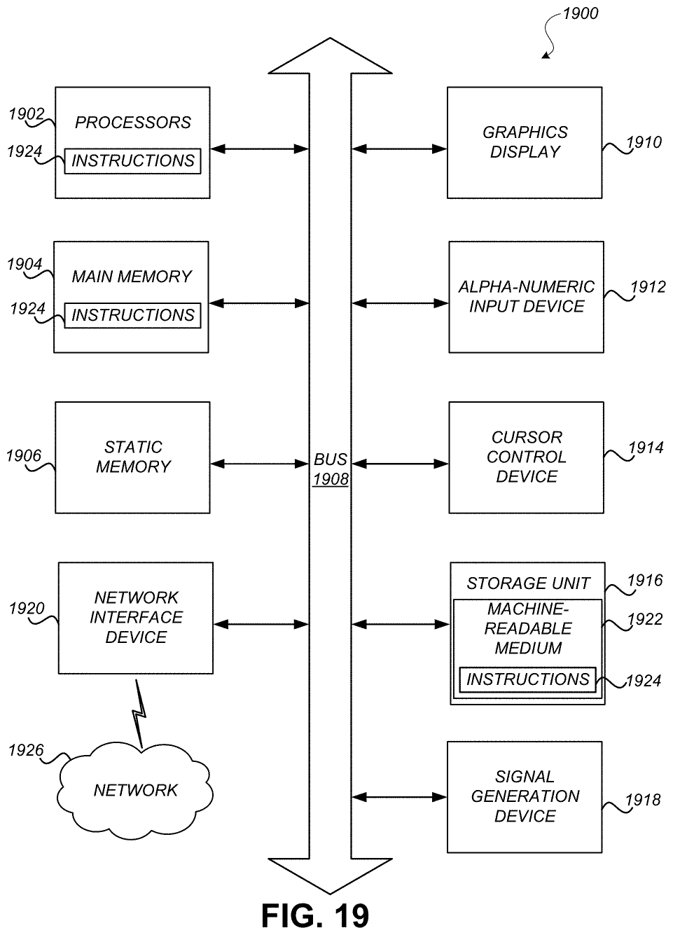

FIG. 19 shows a schematic of an example computer system for implementing various methods of the present invention. In particular, FIG. 19 is a block diagram illustrating components of an example computing machine that is capable of reading instructions from a computer-readable medium and execute them in a processor (or controller). A computer described herein may include a single computing machine shown in FIG. 19, a virtual machine, a distributed computing system that includes multiples nodes of computing machines shown in FIG. 19, or any other suitable arrangement of computing devices.

By way of example, FIG. 19 shows a diagrammatic representation of a computing machine in the example form of a computer system 1900 within which instructions 1924 (e.g., software, program code, or machine code), which may be stored in a computer-readable medium for causing the machine to perform any one or more of the processes discussed herein may be executed. In some embodiments, the computing machine operates as a standalone device or may be connected (e.g., networked) to other machines. In a networked deployment, the machine may operate in the capacity of a server machine or a client machine in a server-client network environment, or as a peer machine in a peer-to-peer (or distributed) network environment.

The structure of a computing machine described in FIG. 19 may correspond to any software, hardware, or combined components (e.g., those shown in FIG. 2 or a processing unit described herein), including but not limited to any engines, modules, computing server, machines that are used to perform one or more processes described herein. While FIG. 19 shows various hardware and software elements, each of the components described herein may include additional or fewer elements.

By way of example, a computing machine may be a personal computer (PC), a tablet PC, a set-top box (STB), a personal digital assistant (PDA), a cellular telephone, a smartphone, a web appliance, a network router, an interne of things (IoT) device, a switch or bridge, or any machine capable of executing instructions 1924 that specify actions to be taken by that machine. Further, while only a single machine is illustrated, the term “machine” and “computer” may also be taken to include any collection of machines that individually or jointly execute instructions 1924 to perform any one or more of the methodologies discussed herein.

The example computer system 1900 includes one or more processors 1902 such as a CPU (central processing unit), a GPU (graphics processing unit), a TPU (tensor processing unit), a DSP (digital signal processor), a system on a chip (SOC), a controller, a state equipment, an application-specific integrated circuit (ASIC), a field-programmable gate array (FPGA), or any combination of these. Parts of the computing system 1900 may also include a memory 1904 that store computer code including instructions 1924 that may cause the processors 1902 to perform certain actions when the instructions are executed, directly or indirectly by the processors 1902. Instructions can be any directions, commands, or orders that may be stored in different forms, such as equipment-readable instructions, programming instructions including source code, and other communication signals and orders. Instructions may be used in a general sense and are not limited to machine-readable codes.

One and more methods described herein improve the operation speed of the processors 1902 and reduces the space required for the memory 1904. For example, the machine learning methods described herein reduces the complexity of the computation of the processors 1902 by applying one or more novel techniques that simplify the steps in training, reaching convergence, and generating results of the processors 1902. The algorithms described herein also reduces the size of the models and datasets to reduce the storage space requirement for memory 1904.

The performance of certain of the operations may be distributed among the more than processors, not only residing within a single machine, but deployed across a number of machines. In some example embodiments, the one or more processors or processor-implemented modules may be located in a single geographic location (e.g., within a home environment, an office environment, or a server farm). In other example embodiments, the one or more processors or processor-implemented modules may be distributed across a number of geographic locations. Even though in the specification or the claims may refer some processes to be performed by a processor, this should be construed to include a joint operation of multiple distributed processors.

The computer system 1900 may include a main memory 1904, and a static memory 1906, which are configured to communicate with each other via a bus 1908. The computer system 1900 may further include a graphics display unit 1910 (e.g., a plasma display panel (PDP), a liquid crystal display (LCD), a projector, or a cathode ray tube (CRT)). The graphics display unit 1910, controlled by the processors 1902, displays a graphical user interface (GUI) to display one or more results and data generated by the processes described herein. The computer system 1900 may also include alphanumeric input device 1912 (e.g., a keyboard), a cursor control device 1914 (e.g., a mouse, a trackball, a joystick, a motion sensor, or other pointing instrument), a storage unit 1916 (a hard drive, a solid state drive, a hybrid drive, a memory disk, etc.), a signal generation device 1918 (e.g., a speaker), and a network interface device 1920, which also are configured to communicate via the bus 1908.

The storage unit 1916 includes a computer-readable medium 1922 on which is stored instructions 1924 embodying any one or more of the methodologies or functions described herein. The instructions 1924 may also reside, completely or at least partially, within the main memory 1904 or within the processor 1902 (e.g., within a processor's cache memory) during execution thereof by the computer system 1900, the main memory 1904 and the processor 1902 also constituting computer-readable media. The instructions 1924 may be transmitted or received over a network 1926 via the network interface device 1920.

While computer-readable medium 1922 is shown in an example embodiment to be a single medium, the term “computer-readable medium” should be taken to include a single medium or multiple media (e.g., a centralized or distributed database, or associated caches and servers) able to store instructions (e.g., instructions 1924). The computer-readable medium may include any medium that is capable of storing instructions (e.g., instructions 1924) for execution by the processors (e.g., processors 1902) and that cause the processors to perform any one or more of the methodologies disclosed herein. The computer-readable medium may include, but not be limited to, data repositories in the form of solid-state memories, optical media, and magnetic media. The computer-readable medium does not include a transitory medium such as a propagating signal or a carrier wave.

### VIII. Additional Considerations

The foregoing description of the embodiments of the invention has been presented for the purpose of illustration; it is not intended to be exhaustive or to limit the invention to the precise forms disclosed. Persons skilled in the relevant art can appreciate that many modifications and variations are possible in light of the above disclosure.

Some portions of this description describe the embodiments of the invention in terms of algorithms and symbolic representations of operations on information. These algorithmic descriptions and representations are commonly used by those skilled in the data processing arts to convey the substance of their work effectively to others skilled in the art. These operations, while described functionally, computationally, or logically, are understood to be implemented by computer programs or equivalent electrical circuits, microcode, or the like. Furthermore, it has also proven convenient at times, to refer to these arrangements of operations as modules, without loss of generality. The described operations and their associated modules may be embodied in software, firmware, hardware, or any combinations thereof.

Any of the steps, operations, or processes described herein may be performed or implemented with one or more hardware or software modules, alone or in combination with other devices. In one embodiment, a software module is implemented with a computer program product including a computer-readable non-transitory medium containing computer program code, which can be executed by a computer processor for performing any or all of the steps, operations, or processes described.

Embodiments of the invention may also relate to a product that is produced by a computing process described herein. Such a product may include information resulting from a computing process, where the information is stored on a non-transitory, tangible computer readable storage medium and may include any embodiment of a computer program product or other data combination described herein.

Finally, the language used in the specification has been principally selected for readability and instructional purposes, and it may not have been selected to delineate or circumscribe the inventive subject matter. It is therefore intended that the scope of the invention be limited not by this detailed description, but rather by any claims that issue on an application based hereon. Accordingly, the disclosure of the embodiments of the invention is intended to be illustrative, but not limiting, of the scope of the invention, which is set forth in the following claims.

What is claimed is:

1. A method for processing sequencing data of ribonucleic acid (RNA) molecules from a test sample, the method comprising:
obtaining a plurality of sequence reads each derived from a RNA molecule obtained from the test sample;
filtering the plurality of sequence reads;
identifying one or more candidate variants from the filtered plurality of sequence reads;
determining a quality score for each of the identified one or more candidate variants, the quality score indicating a likelihood that the candidate variant is a false positive detection of a mutation in the RNA molecule; and
outputting the one or more candidate variants having a quality score greater than a threshold quality score.

2. The method of claim 1, wherein obtaining the plurality of sequence reads comprises:
obtaining the test sample from an individual, the test sample comprising a plurality of RNA molecules;
preparing a RNA sequencing library from the plurality of RNA molecules; and
generating the plurality of sequence reads from the RNA sequencing library.

3. The method of claim 2, wherein the sequencing library is enriched for one or more targeted RNA molecules prior to obtaining the plurality of sequence reads.

4. The method of claim 2, wherein the plurality of sequence reads are obtained using next-generation sequencing of the RNA sequencing library.

5. The method of claim 2, wherein the plurality of RNA molecules are RNA transcripts, wherein the RNA transcripts are messenger RNA, transfer RNA, or ribosomal RNA.

6. The method of claim 1, wherein filtering the plurality of sequence reads comprises:
filtering at least one sequence read of the plurality of sequence reads having a least a threshold number of continuous nucleotide base mutations;
filtering at least one sequence read of the plurality of sequence reads having at least a threshold depth; and/or
filtering out a number of leading nucleotide bases of at least one sequence read of the plurality of sequence reads.

7. The method of claim 6, wherein the threshold number of continuous nucleotide base mutations is at least three, the threshold depth is at least 50,000, or the number of leading nucleotide bases is six.

8. The method of claim 1, wherein the threshold quality score is determined by performing calibration using a plurality of calibration samples, each calibration sample including one or more control RNA molecules and a plurality of RNA molecules from one or more individuals.

9. The method of claim 8, wherein the one or more control RNA molecules are associated with External RNA Controls Consortium (ERCC) Spike-In Control Mixes, and wherein the one or more individuals are healthy.

10. The method of claim 8, wherein performing the calibration using calibration samples comprises:
for each of the plurality of calibration samples:
determining a depth of the calibration sample; and
determining a sensitivity of the calibration sample, the sensitivity indicating a likelihood of detecting false positive mutations in the calibration sample.

11. The method of claim 1, wherein determining the quality score for a candidate variant comprises:
accessing a plurality of parameters including a dispersion parameter r and a mean rate parameter m specific to the candidate variant, the r and m having been derived using a model;
inputting read information of the plurality of sequence reads into a function parameterized by the plurality of parameters; and
determining the quality score for the candidate variant using an output of the function based on the input read information.

12. The method of claim 11, wherein the plurality of parameters represent mean and shape parameters of a gamma distribution, and wherein the function is a negative binomial based on the plurality of sequence reads and the plurality of parameters.

13. The method of claim 11, wherein the plurality of parameters represent parameters of a distribution that encodes an uncertainty level of nucleotide mutations with respect to a given position of a sequence read.

14. The method of claim 13, wherein a gamma distribution is one component of a mixture of the distribution.

15. The method of claim 11, wherein the plurality of parameters are derived from a training sample of sequence reads from a plurality of healthy individuals.

16. The method of claim 15, wherein the training sample excludes a subset of the sequence reads from the plurality of healthy individuals based on filtering criteria when the sequence reads that have (i) a depth less than a threshold value or (ii) an allele frequency greater than a threshold frequency.

17. The method of claim 11, wherein the plurality of parameters are derived using a Bayesian Hierarchical model.

18. The method of claim 17, wherein the Bayesian Hierarchical model includes a multinomial distribution grouping positions of sequence reads into latent classes.

19. The method of claim 17, wherein the Bayesian Hierarchical model includes fixed covariates unrelated to training samples from healthy individuals, wherein the covariates are based on a plurality of nucleotides adjacent to a given position of a sequence read, or wherein the covariates are based on a level of uniqueness of a given sequence read relative to a target region of a genome.

20. The method of claim 17, wherein the Bayesian Hierarchical model is estimated using a Markov chain Monte Carlo method.

21. The method of claim 20, wherein the Markov chain Monte Carlo method uses a Metropolis-Hastings algorithm, a Gibbs sampling algorithm, or Hamiltonian mechanics.

22. The method of claim 11, wherein the sequence read information includes a depth d of the plurality of sequence reads, the function parameterized by m·d.

23. The method of claim 11, wherein the quality score is a Phred-scaled likelihood.

24. The method of claim 11, further comprising:
determining that the candidate variant is a false positive mutation by comparing the quality score to a threshold quality score.

25. The method of claim 24, wherein the candidate variant is a single nucleotide variant.

26. The method of claim 25, wherein the model encodes noise levels of nucleotide mutations for one base of A, U, C, and G to each of the other three bases.

27. The method of claim 11, wherein the candidate variant is an insertion or deletion of at least one nucleotide.

28. The method of claim 27, wherein the model includes a distribution of lengths of insertions or deletions.

29. The method of claim 28, wherein the model separates an inference for determining a likelihood of an alternate allele from an inference for determining a length of the alternate allele using the distribution of lengths.

30. The method of claim 28, wherein the distribution of lengths comprises a multinomial with Dirichlet prior, wherein the Dirichlet prior on the multinomial distribution of lengths is determined by covariates of anchor positions of a genome.

31. The method of claim 27, wherein the model includes a distribution ω determined based on covariates.

32. The method of claim 27, wherein the model includes a distribution φ determined based on covariates and anchor positions of a genome.

33. The method of claim 27, wherein the model includes a multinomial distribution grouping lengths of insertions or deletions at anchor positions of sequence reads into latent classes.

34. The method of claim 27, wherein an expected mean total count of insertions or deletions at a given anchor position is modeled by a distribution based on covariates and anchor positions of a genome.

35. The method of claim 1, wherein the plurality of sequence reads are obtained from sequencing RNA molecules from a sample of blood, whole blood, plasma, serum, urine, cerebrospinal fluid, fecal, saliva, tears, a tissue biopsy, pleural fluid, pericardial fluid, or peritoneal fluid of an individual.

36. The method of claim 1, wherein the plurality of sequence reads are obtained from sequencing RNA molecules from a tumor biopsy.

37. The method of claim 1, wherein the plurality of sequence reads are obtained from sequencing a RNA molecules from an isolate of cells from blood, the isolate of cells including at least buffy coat white blood cells or CD4+ cells.

38. A system comprising a computer processor and a memory, the memory storing computer program instructions that when executed by the computer processor cause the processor to:
obtain a plurality of sequence reads each derived from a RNA molecule obtained from the test sample;
filter the plurality of sequence reads;
identify one or more candidate variants from the filtered plurality of sequence reads;
determine a quality score for each of the identified one or more candidate variants, the quality score indicating a likelihood that the candidate variant is a false positive detection of a mutation in the RNA molecule; and
output the one or more candidate variants having a quality score greater than a threshold quality score.

39. A non-transitory computer-readable storage medium storing instructions that when executed by a processor cause the processor to:
obtain a plurality of sequence reads each derived from a RNA molecule obtained from the test sample;
filter the plurality of sequence reads;
identify one or more candidate variants from the filtered plurality of sequence reads;
determine a quality score for each of the identified one or more candidate variants, the quality score indicating a likelihood that the candidate variant is a false positive detection of a mutation in the RNA molecule; and
output the one or more candidate variants having a quality score greater than a threshold quality score.
